(19) United States
(12) Patent Application Publication
(10) Pub. No.: US 2026/0355403 A1
(43) Pub. Date: Nov. 12, 2026

(54) Medical Waste Collection And Disposal Systems

(71) Applicant: Stryker Corporation, Portage, MI (US)

(72) Inventors: Brian MacLachlan, Norton Shores, MI (US); Brooke Mason, Mattawan, MI (US); Glen D. Rocque, Portage, MI (US); Gaurav Retwal, Faridabad (IN); Troy E. Durnell, Lakewood, NY (US); Ross Nave, Kalamazoo, MI (US)

(73) Assignee: Stryker Corporation, Portage, MI (US)

(21) Appl. No.: 19/124,677

(22) PCT Filed: Oct. 25, 2023
(86) PCT No.: PCT/US2023/035934

§ 371 Date: Apr. 25, 2025

Related U.S. Application Data
(60) Provisional application No. 63/419,012, filed on Oct. 25, 2022.

(51) Int. Cl. A61M 1/00

(52) U.S. Cl. CPC A61M 1/63

(57) ABSTRACT

A medical waste collection unit including collected medical waste is docked with a docking station to form a fluid connection and a first data connection therebetween. A cleaning cycle is performed on the medical waste collection unit in which cleaning fluid is supplied from the docking station and sprayed within the waste collection unit, and in which waste material is discharged from the waste collection unit through the docking station. A status update indicative of a docked status of the waste collection unit is communicated from the docking station to a remote processing system over a second data connection, and responsive notification data is received at the docking station from the remote processing system. The notification data indicates a maintenance notification to be displayed on the medical waste collection unit, and is triggered by the docking station for display on the waste collection unit over the first data connection.

### RELATED APPLICATIONS

The present Application claims priority to and all the benefits of U.S. Provisional Patent Application No. 63/419012, filed Oct. 25, 2022, the contents of which are hereby incorporated by reference herein in their entirety.

### BACKGROUND

Waste collection devices are used in healthcare facilities to collect waste material during medical procedures. A healthcare facility will frequently employ several of such devices to accommodate various procedures occurring at different parts of the facility. Routine maintenance and proper allocation of these devices are needed to ensure their efficient operation and utilization.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of maintaining a medical waste collection unit including a canister for collecting medical waste produced during a surgical procedure. The method includes transporting the medical waste collection unit with the collected medical waste to a docking station, the docking station including a supply line coupled to a first interface for establishing a fluid supply connection with the medical waste collection unit when docked with the docking station, a discharge line coupled to a second interface for establishing a fluid discharge connection with the medical waste collection unit when docked with the docking station, a first communications interface for establishing a first data connection with the medical waste collection unit when docked to the docking station, and a second communications interface for establishing a second data connection with a remote processing system. The method also includes docking the medical waste collection unit with the docking station to form the fluid supply connection, the fluid discharge connection, and the first data connection. The method also includes performing a cleaning cycle on the medical waste collection unit in which cleaning fluid is supplied from the docking station supply line to the medical waste collection unit through the fluid supply connection and sprayed into the canister, and in which waste material is discharged from the canister to the docking station discharge line through the fluid discharge connection. The method also includes communicating, from the docking station and through the second data connection, a status update to the remote processing system indicative of a docked status of the medical waste collection unit. The method also includes receiving, at the docking station and through the second data connection, notification data from the remote processing system that is responsive to the status update, the notification data indicating a maintenance notification to be displayed on the medical waste collection unit. The method also includes triggering, by the docking station and through the first data connection, the medical waste collection unit to display a maintenance notification based on the notification data. Other implementations of this aspect include corresponding computer systems, apparatus, and computer programs recorded on one or more computer storage devices, each configured to perform the actions of the methods.

One general aspect includes a method of maintaining a medical waste collection unit including a canister for holding medical waste produced during a surgical procedure. The method includes transporting the medical waste collection unit with the collected medical waste to a docking station, the docking station including a supply line coupled to a first interface for establishing a fluid supply connection with the medical waste collection unit when docked with the docking station, a discharge line coupled to a second interface for establishing a fluid discharge connection with the medical waste collection unit when docked with the docking station. The method also includes docking the medical waste collection unit with the docking station to form the fluid supply connection and the fluid discharge connection. The method also includes performing a cleaning cycle in which cleaning fluid is supplied from the docking station supply line to the medical waste collection unit through the fluid supply connection and sprayed into the canister, and in which waste material is discharged from the canister to the docking station discharge line through the fluid discharge connection. The method also includes tracking, by one or more controllers, a cleaning cycle metric for determining whether to trigger a maintenance notification indicating to perform the cleaning cycle on the medical waste collection unit with the docking station. The method also includes resetting, by the one or more controllers, the cleaning cycle metric in response to the cleaning cycle being performed on the medical waste collection unit by the docking station. The method also includes determining, by the one or more controllers, a notification threshold based on operational data of the medical waste collection unit, the operational data indicating surgical procedure data of the medical waste collection unit and/or cleaning cycle data of the medical waste collection unit. The method also includes triggering, by the one or more controllers, display of the maintenance notification on the medical waste collection unit based on the cleaning cycle metric and the notification threshold. Other implementations of this aspect include corresponding computer systems, apparatus, and computer programs recorded on one or more computer storage devices, each configured to perform the actions of the methods.

One general aspect includes a method of managing a fleet of medical waste collection units each including a canister for holding medical waste. The method includes receiving, by a processing system remote from the medical waste collection units, first usage data from a first of the medical waste collection units indicative of a duration in which the first medical waste collection unit is operated to draw medical waste into the canister of the first medical waste collection unit by a vacuum source. The method also includes receiving, by the remote processing system, second usage data from a second of the medical waste collection units indicative of a duration in which the second medical waste collection unit is operated to draw medical waste into the canister of the second medical waste collection unit by a vacuum source. The method also includes comparing, by the remote processing system, the first usage data with the second usage data to determine relative usage data for the first medical waste collection unit and the second medical waste collection unit. The method also includes comparing, by the remote processing system, the relative usage data to a preset relative usage threshold. The method also includes based on the comparison, triggering a usage notification on a least one of the first and second medical waste collection units. Other implementations of this aspect include corresponding computer systems, apparatus, and computer programs recorded on one or more computer storage devices, each configured to perform the actions of the methods.

One general aspect includes a method of managing a medical waste collection unit including a canister for holding medical waste and an aerosol evacuation unit including a filter. The method includes tracking, by one or more controllers, a filter metric for determining whether to indicate to replace the filter based on operational data of the medical collection unit, the operational data indicating surgical procedure data of the medical waste collection unit. The method also includes resetting, by the one or more controllers, the filter metric in response to a filter change event. The method also includes triggering, by the one or more controllers, display of a maintenance notification on a display of a personal computing device remote from the medical waste collection unit that directs a user to replace the filter based on the filter metric. Other implementations of this aspect include corresponding computer systems, apparatus, and computer programs recorded on one or more computer storage devices, each configured to perform the actions of the methods.

One general aspect includes a method for maintaining a medical waste collection unit including a canister for collecting medical waste material produced during a surgical procedure. The method includes capturing, by an imaging device supported by a device cradle coupled to the canister, a video feed of the canister and the waste material disposed in the canister as a vacuum source of the medical waste collection unit is drawing waste material into the canister. The method also includes analyzing, by one or more controllers, image frames of the video feed to determine vision data indicating at least one a degree of occlusion, a composition of the waste material, and a storage time of the waste material. The method also includes tracking, by the one or more controllers, a metric for determining whether to trigger a maintenance notification on the medical waste collection unit. The method also includes determining, by the one or more controllers, a notification threshold based on the vision data. The method also includes triggering, by the one or more controllers, the maintenance notification on the medical waste collection unit based on the notification threshold and the metric. Other implementations of this aspect include corresponding computer systems, apparatus, and computer programs recorded on one or more computer storage devices, each configured to perform the actions of the methods.

### BRIEF DESCRIPTION OF THE DRAWINGS

FIG. 1A illustrates an exemplary medical waste collection and disposal system including a medical waste collection unit for collecting medical waste during a surgical procedure and a docking station for emptying and cleaning the medical waste collection unit.

FIG. 1B illustrates an exemplary aerosol evacuation unit of the medical waste collection unit.

FIG. 2 illustrates an exemplary system for maintaining a group of medical waste collection units that may be employed by a health care system.

FIG. 3 illustrates another exemplary system for maintaining a group of medical waste collection units that may be employed by a health care system.

FIG. 4 illustrates the medical waste collection unit with a device cradle coupled to the medical waste collection unit and positioned to removably receive an imaging device for capturing an image of the waste canister.

FIG. 5 illustrates an exemplary method of maintaining a fleet of medical waste collection units that may be employed by a health care facility or system.

FIGS. 6A and 6B illustrate a graphical user interface (UI) that may be generated during the method of FIG. 5 FIG. 7 illustrates another exemplary method for maintaining a fleet of medical waste collection units that may be employed by a healthcare facility or system.

FIG. 8 illustrates an exemplary screen of a graphical user interface (GUI) that may be generated by the system of FIG. 2 or FIG. 3 to provide information about the fleet of medical waste collection units and associated docking stations.

FIG. 9 illustrates another exemplary screen of the GUI that provides a detailed listing of the fleet of medical waste collection units and associated docking stations.

FIG. 10 illustrates another exemplary screen of the GUI to display a status of a given medical waste collection unit.

FIG. 11 illustrates another exemplary screen of the GUI to display a status of a given docking station.

FIG. 12 illustrates another exemplary screen of the GUI to display a summary of the fleet of medical waste collection units.

FIG. 13 illustrates another exemplary screen of the GUI that provides manifold usage data for the fleet of medical waste collection units.

FIG. 14 illustrates another exemplary screen of the GUI that provides error code data for the fleet of medical waste collection units.

FIG. 15 illustrates another exemplary screen of the GUI that provides filter data for the fleet of medical waste collection units.

FIGS. 16A and 16B illustrates exemplary screens of the GUI that provide return on investment data for the fleet of medical waste collection units.

FIG. 17 illustrates another exemplary screen of the GUI that provides vacuum pump runtime data for the fleet of medical waste collection units.

FIG. 18 illustrates another exemplary screen of the GUI that provides a summary of the docking stations associated with the fleet of medical waste collection units.

FIG. 19 illustrates another exemplary screen of the GUI that provides cleaning cycle data for the fleet of medical waste collection units.

FIG. 20 illustrates another exemplary screen of the GUI that provides facility waster data for the fleet of medical waste collection units.

### DETAILED DESCRIPTION

Aspects of this disclosure generally relate to operating and maintaining mobile waste collection units, also referred to herein as rovers, utilized by a health care facility or system to collect waste material during medical procedures. A given health care facility or system may employ several rovers that vary in location and usage, and the optimal maintenance schedules of the rovers may also vary as a result. Management of the rovers is thus a difficult and complex task for administrators and clinical users alike, and if inadequately conducted can lead to suboptimal rover performance, increased service calls, and reduced rover life.

As one non-limiting example, following a surgical procedure with a given rover, the rover may be docked to a docking station to drain the collected waste material and perform a cleaning cycle on the rover. The docking station may be configured to perform varying types of cleaning cycles (e.g., quick wash cleaning cycle, normal wash cleaning cycle, extended wash cleaning cycle) depending on the level of “cleaning” that is needed. To thoroughly clean and ensure proper functioning of the rover, extended cleaning cycles should be regularly performed, and components of the rover should be regularly evaluated and replaced. In the context of a fleet of rovers with varying usage and location, however, it is difficult to determine and ensure compliance with optimal maintenance schedules, including the performance of extended cleaning cycles, for each rover, which may vary as a function of the specific usage of the rover, and to efficiently manage rover usage as a function of the estimated life and maintenance schedules of the rovers as a group.

The present disclosure provides systems and methods for overcoming these and other challenges arising from the use and maintenance of a fleet of rovers with varying usage and locations. As an example, systems and methods described herein may be configured to implement a centralized management and notification service that monitors the specific activity of each rover of a fleet, as well the activity of the fleet as a whole, to dynamically update maintenance and usage schedules for the fleet in real time, promoting both the longevity of the individual rovers and efficient use of the fleet. The systems and methods may also be configured to generate a notification to the person best suited to take action based on such monitoring, and to aggregate data from multiple rovers to enable fast and informed decision making.

The systems and methods described herein are unconventional in the technical field of medical waste collection devices. Managing the use and maintenance of waste collection devices within a healthcare facility or system has conventionally been performed by noting a cleaning history of each device on whiteboards or other physical tracking sheet, such as by noting the date, time, and device ID whenever a cleaning cycle is performed. However, such a technique is inefficient in the context of a fleet of rovers being used in varying locations, with varying frequencies, and for varying types of procedures within a healthcare facility or system. Specifically, due to the transient nature of such devices, the noted history may become quickly outdated or not be considered as medical personnel may be delayed in updating, reviewing, and digesting such information between procedures. By implementing a system that considers individual rover activity and the fleet as a whole to dynamically control how the rovers are operated in real time, such as by leveraging nearby mobile devices or the docking stations to which the rovers are couplable for cleaning as edge devices for communicating rover operational data to a central location, aspects of the present disclosure help to overcome the above challenges while minimizing any impact to the configuration or operation of the rover.

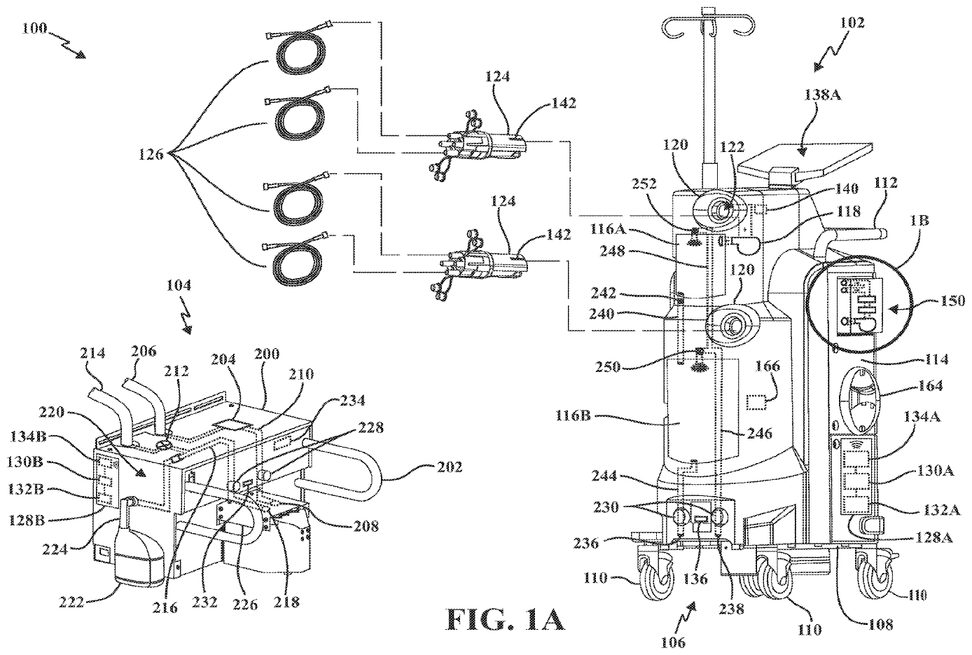

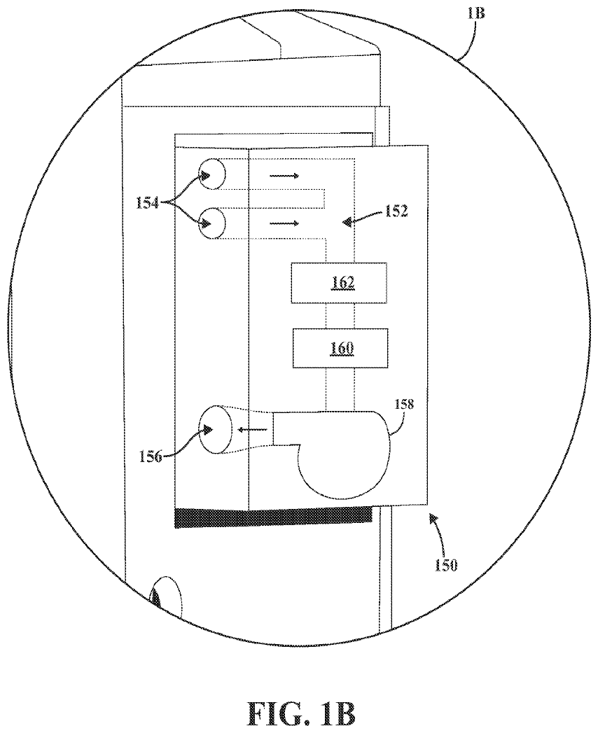

FIG. 1A illustrates a medical waste collection and disposal system 100 for collecting and disposing of waste material produced during medical procedures (e.g., surgical procedures) performed in a health care facility such as a hospital. The waste material may include bodily fluids, body tissues, irrigation liquids, and/or other materials that may be generated during various medical procedures. Often times, medical procedures require large amounts of saline and/or other irrigation liquids for irrigating an anatomical site. As a result, the system 100 may be capable of handling large amounts of waste material.

The system 100 may comprise a mobile waste collection unit 102, also referred to herein as a rover, and a generally fixed docking station 104. The rover 102 may be configured to collect the waste material generated during medical procedures. The docking station 104 may function as a unit through which the waste material collected by the rover 102 is discharged for treatment. The docking station 104 may also function to clean the rover 102, as explained in more detail below. During use, the rover 102 may collect and store the waste material on-board until such a time as a user is ready to off-load and dispose of the waste material. In some implementations, the rover 102 may be capable of storing waste material from a series of different medical procedures during the course of a day or across several days, without requiring off-loading of the waste material. Once the waste material either fills the rover 102, or a user is ready to dispose of the waste material, the rover 102 may be wheeled to the docking station 104 by the user. At the docking station 104, the waste material may be emptied from the rover 102 to a waste drain or treatment area, and the rover 102 may be cleaned for further use.

The rover 102 may include a cart 106 that provides mobility to the rover 102. The cart 106 may include a cart base 108 and a plurality of wheels 110 mounted to a bottom of the cart base 108. A handle 112 may be mounted to a vertical chassis 114 extending from the cart base 108 to facilitate movement of the rover 102 between use areas, and between the use areas and the docking station 104. Thus, users can move the rover 102 around the health care facility to collect waste material generated during medical procedures performed in different locations throughout the health care facility or system.

The rover 102 may also include one or more waste canisters 116, such as an upper waste canister 116A and a lower waste canister 116B, to collect and temporarily store the waste material during use. One or more vacuum pumps 118 may be supported on the cart base 108, and may be configured to draw suction on the waste canister(s) 116 through one or more vacuum lines.

Suitable construction and operation of several subsystems of the rover 102 may be disclosed in commonly owned United States Patent Publication No. 2005/0171495, International Publication No. WO 2007/070570, and International Publication No. WO 2014/066337, the disclosures of which are hereby incorporated by reference herein in their entirety. Suitable construction and operation of several subsystems of the rover 102 may also be disclosed in commonly owned International Publication No. WO 2017/112684, published Jun. 29, 2017, the disclosure of which is hereby incorporated by reference herein in its entirety.

The rover 102 may further include at least one receiver 120 supported on the cart base 108. The receiver(s) 120 may each define an opening 122 dimensioned to removably receive at least a portion of a manifold 124, such as a surgical waste collection manifold 124. FIG. 1 illustrates two receivers 120 each associated with a different one of the waste canisters 116. In alternative implementations, a single receiver 120 may be provided for all of the waste canisters 116. Each receiver 120 may include a suction inlet configured to be arranged in fluid communication with the waste canister(s) 116 associated with the receiver 120. A suction path may thus be established from suction tube(s) 126 disposed in a surgical site to the waste canister(s) 116 through the manifold(s) 124 when removably inserted into the receiver(s) 120, to thereby suction waste material from the surgical site to the waste canister(s) 116. The manifold(s) 124 may be configured to prevent waste material from passing back through the suction tubes 126 once collected by the rover 102. Each manifold 124 may also function to filter the waste material received from the suction tubes, such as for large particles so as to prevent clogging. Some versions of the manifold(s) 124 may further include a compartment for collecting and holding a tissue specimen, such as for subsequent analysis.

The rover 102 may include a rover controller 128A configured to control actuation of the rover 102. To this end, the rover controller 128A may be in communication with and configured to regulate the on/off operation of the vacuum pump(s) 118, and also to regulate the extent of operation of the vacuum pump(s) 118 so as to control the vacuum flow through the manifold(s) 124. In some implementations, the rover controller 128A may include a processor 130A, memory 132A, and a communications device 134A. The processor 130A may include one or more devices selected from microprocessors, micro-controllers, digital signal processors, microcomputers, central processing units, field programmable gate arrays, programmable logic devices, state machines, logic circuits, analog circuits, digital circuits, and/or any other devices that manipulate signals (analog or digital) based on operational instructions stored in the memory 132A. The memory 132A may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, and/or any other device capable of storing information. The memory 132A may also include one or more persistent data storage devices including, but not limited to, a hard drive, optical drive, tape drive, non-volatile solid state device, and/or any other device capable of persistently storing information.

The processor 130A may be configured to implement the functions, features, and processes of the rover controller 128A described herein. More specifically, the processor 130A may operate under control of software embodied by computer-executable instructions residing in the memory 132A, with the computer-executable instructions being configured, upon execution by the processor 130A, to cause the processor 130A to implement the functions, features, and processes of the rover controller 128A described herein. The computer-executable instructions may be compiled or interpreted from a variety of programming languages and/or technologies, including, but not limited to, and either alone or in combination, Java, C, C++, C#, Objective C, Fortran, Pascal, Java Script, Python, Perl, and PL/SQL. The memory 132A may also store data that may be accessed by the rover controller 128A, or more particularly read by the processor 130A, to facilitate the functions, features, and processes of the rover controller 128A described herein.

The communications device 134A may provide a machine interface that facilitates data communication between the rover controller 128A and one or more external systems and devices, such as other rovers 102, the docking station 104, a remote terminal, a nearby mobile device, and a remote server (e.g., cloud service). The communications device 134A may incorporate one or more wired and/or wireless technologies to facilitate such communication, including, but not limited to, Ethernet, USB, Wi-Fi, Bluetooth, and cellular. In some instances, given the transient nature of the rover 102, the communications device 134A of the rover controller 128A may be limited to relatively short-range and/or direct connection communication technologies, or more particularly relatively short-range and/or direct connection wireless communication technologies, such as proximity-based wireless communication technologies (e.g., RFID, NFC), Bluetooth, Wi-Fi Direct, Zigbee, or line-of-site based wireless communication technologies (e.g., infrared (IR)). In other words, the communications device 134A may lack relatively long range communication capabilities, such as cellular and Wi-Fi, that allow for connection with remote systems and devices over one or more computer networks. In some implementations, the communications device 134A may be coupled to an IR transceiver 136 facing outwardly the rover 102 to facilitate wireless communication with other devices, such as the docking station 104, using IR transmissions.

The rover 102 may also include a user interface 138A in operable communication with the rover controller 128A. The user interface 138A may be configured to present operational data relating to the rover 102 to a user, and to accept user input for controlling operation of the rover 102. For example and without limitation, the user interface 138A may include a display, one or more light emitting diodes (LEDs), and/or a speaker for providing operational data relating to the rover 102 to a user. Additionally, the user interface 138A may include a touch screen associated with the display to present a graphical user interface (GUI) with user-selectable elements by which a user may enter commands to regulate operation of the rover 102, and/or may include one or more mechanically operable input devices such as buttons or a dial by which a user may enter commands to regulate operation of the rover 102. In some instances, the user interface 138A may also include a microphone for receiving voice commands from a user. In some instances, the user interface 138A may be provided by a tablet with a display that is removably mountable to the vertical chasses 114 of the rover 102, and in communication with the rover controller 128A via the communications device 134A.

The rover 102 may further include a reader 140 positioned adjacent each receiver 120. Each reader 140 may be configured to communicate with an RFID tag 142 of a manifold 124 when inserted into the associated receiver 120. The RFID tag 142 may be coupled to a surface, such as an internal or external surface, of the manifold 124. The rover controller 128A may be in communication with each reader 140, and may be configured to instruct each reader 140 to periodically emit a basic interrogation signal for an RFID tag 142. If a manifold 124 is not seated in a given receiver 120, then the reader 140 associated with the receiver 120 may not receive a response to the basic interrogation signal. The rover controller 128A may be configured inhibit activation of the vacuum pump 118 associated with a given receiver 120 if a manifold 124 is not seated in the receiver 120, or more specifically if a compatible manifold 124 is not seated in the receiver 120, which may be determined based on data read from the RFID tag 142 of a compatible manifold 124 when the manifold 124 is seated in the receiver 120.

The rover 102 may also include an aerosol evacuation system 150, an enlarged view of which is provided in FIG. 1B. The aerosol evacuation system 150 may be utilized for removing aerosols, such as smoke, from a fluid, such as air, during a surgical operation. The aerosol evacuation system 150 may include a conduit 152 and one or more inlets 154 in fluid communication with the conduit 152, such that fluid may be drawn into the conduit 152 via the inlet(s) 154. The aerosol evacuation system 150 may further include an outlet 156 through which fluid is exhausted from the conduit 152. The fluid drawn through the inlet(s) 154 may be air along with aerosols such as smoke that are generated during a medical procedure. A blower 158 of the aerosol evacuation system 150 may be in fluid communication with the conduit 152 for drawing the fluid into the inlet(s) 154 and out the outlet 156 when the blower 158 is activated.

The aerosol evacuation system 150 may also include a filter 160 in fluid communication with the conduit 152. The filter 160 may be configured to filter one or more aerosols from the fluid running through the conduit 152 such that “clean” air is exhausted from the outlet 156. For instance, the filter 160 may include a filter, such as an ULPA filter, for filtering smoke generated during a surgical procedure. The filter 160 may preferably be supported by a filter housing including a filter enclosure and a filter cap to form a replaceable unit.

The aerosol evacuation system 150 may further include an aerosol sensor 162 in fluid communication with the conduit 152 and electrically connected to the rover controller 128A. The aerosol sensor 162 may be disposed inline with the conduit 152 such that the fluid flowing through the conduit 152 may be sensed before passing through the filter 160. Said another way, the aerosol sensor 162 may be upstream from the filter 160. The aerosol sensor 162 may be disposed within the filter enclosure, and may likewise be replaceable along with the filter 160 to ensure accurate readings.

The aerosol sensor 162 may be configured to sense a presence and/or an amount of one or more aerosols, such as smoke, traveling through the conduit 152, and to generate one or more sensor signals indicative of the presence and/or amount of each aerosol. The sensor signal(s) may be communicated to the rover controller 128A. Based on the presence and/or amount of each aerosol indicated by the sensor signal(s), the rover controller 128A may be configured to track compliance with certain safety recommendations (e.g., surgical smoke evacuation), track a remaining life of the filter 160, and/or control operation of the blower 158. For example, the rover controller 128A may be configured to perform one of more of the above operations by tracking a duration in which aerosol(s) are indicated present by the sensor signal(s).

As one non-limiting example, the aerosol sensor 162 may include at least one IR lamp for emitting IR light into the conduit 152, and may include at least one IR detector for sensing reflections of the emitted IR light by the fluid moving through the conduit 152. The aerosol sensor 162 may then generate one or more sensor signals corresponding to the sensed reflections, which in turn may be indicative of the presence and/or amount of one or more aerosols moving through the conduit 152. The rover controller 128A may then be configured to evaluate the sensor signal(s) to determine the presence and/or amount of one or more aerosols moving through the conduit 152. The aerosol sensor 162 may alternatively be realized as another type of sensor, such as electromagnetic sensor configured to sense an electromagnetic field from an aerosol detection cable disposed within the conduit 152 or near the surgical site, the aerosol detection cable configured to emit a varying electromagnetic field as a function of the presence and/or amount of an aerosol such as smoke adjacent the cable.

Based on the evaluation of the sensor signal(s), the rover controller 128A may be configured to vary operation of the blower 158. For instance, when the sensor signal(s) indicate an absence of monitored aerosol(s) in the conduit 152, the rover controller 128A may be configured to operate the blower 158 at a relatively low power level. That is, the blower 158 may be operated to provide just enough suction to draw fluid into the conduit 152, such that one or more aerosols can be sensed by the aerosol sensor 162 if present.

During operation of the rover 102, the rover controller 128A may receive one or more sensor signals representing the presence and/or an amount of each of one or more aerosols sensed in the conduit 152. When, based on the sensor signal(s), the rover controller 128A detects one or more aerosols in the conduit 152, or more particularly detects that the amount of an aerosol in the conduit 152 is greater than a threshold amount, the rover controller 128A may be configured to increase operation of the blower 158 to a relatively high power level. This relatively high power level may be configured to quickly accelerate the operation of the blower 158 (e.g., quickly accelerate rotation of a fan within the blower 158). After operating the blower 158 at the relatively high power level, the rover controller 128A may be configured to decrease a power level of the blower 158, such as to a mid-power level that is greater than the relatively low power level and less than the relatively high power level.

When operating at the mid-power level, the blower 158 may generate more suction at the inlet(s) 154 than when the blower 158 is operating at the relatively low power level. This enables aerosols such as smoke to be quickly evacuated from the surgical site and filtered by the filter 160. While the blower 158 is operating at the mid-power level, the aerosol sensor 162 may be configured to continue evaluating the fluid passing through the conduit 152 for one or more aerosols. Responsive to the sensor signal(s) indicating that the presence of each of the monitored aerosols in the conduit 152 is less than the threshold amount or not detected, the rover controller 128A may be configured to return the blower 158 to the relatively low power level, and continue to evaluate the sensor signal(s) for the presence of the monitored aerosols as described above.

By operating the blower 158 at the relatively low power level, noise caused by the blower 158 is noticeably reduced, which helps maintain a more peaceful environment when delicate surgical operations are being performed. However, by quickly ramping up to the higher power levels, the aerosol evacuation system 150 may retain the performance level needed to quickly evacuate aerosol(s) from the surgical area. The above power levels of the blower 158 may be configurable by a user via the user interface 138A. Additionally or alternatively, the blower 158 may be configured to operate at a constant power level throughout a surgical operation, which may be varied by the user via the user interface 138A.

Referring back to FIG. 1A, the rover 102 may also include one or more particle filters 164, such as a HEPA filter, in fluid communication with the waste canister(s) 116 for filtering fluid (e.g., air) suctioned into the waste canister(s) 116 during collection of waste material from a surgical site. Similar to the filter 160, the particle filter(s) 164 may also be replaceable.

The rover 102 may also include one or more sensors 166 in communication with the rover controller 128A that are configured to generate operational data relating to use of the rover 102. For example and without limitation, the sensor(s) 166 may include a fluid measuring subsystem arranged to measure the volume of waste material received in the rover 102, or more particularly in each of the waste canister(s) 116 through the manifold(s) 124, during a medical procedure, and/or the volume of cleaning fluid received in the rover 102, or more particularly in each of the waste canister(s) 116, during a cleaning cycle. Additionally or alternatively, the sensor(s) 166 may include one or more flow rate sensors to measure a rate of flow of waste material passing through the manifold(s) 124 during a medical procedure and/or of cleaning fluid passing through the rover 102 during a cleaning cycle. Additionally or alternatively, the sensor(s) 166 may include one or more blood concentration sensors for measuring a concentration of blood within the waste material passing through the manifold(s) 124 during a medical procedure. Additionally or alternatively, the sensor(s) 166 may include one or more temperature sensors for measuring the temperature of cleaning fluid passing through the rover 102 during a cleaning cycle. Exemplary measurement or monitoring devices are disclosed in commonly-owned International Application No. PCT/US 2021/058891, filed Nov. 11, 2021, the entire contents of which are hereby incorporated herein by reference.

As described above, the rover 102 may be periodically docked with the docking station 104 to empty and/or clean the waste canister(s) 116. Referring again to FIG. 1A, the docking station 104 may include a metal cabinet 200 generally in the shape of a box. Guide rails 202 may extend from a front of the cabinet 200 to guide the rover 102 when docking to the docking station 104. An off-load pump 204 may be disposed inside the cabinet 200, and may be connected to a drain line 206 and to a waste coupler 208 through a waste line 210. The off-load pump 204 may be configured to pump waste material from the rover 102 to the drain line 206 through the waste coupler 208 and waste line 210 when the rover 102 is docked to the docking station 104. The drain line 206 may extend from the off-load pump 204 to a waste site.

A water valve 212 may also be disposed inside the cabinet 200. The water valve 212 may be connected to a water source in the health care facility via a supply line 214. For example, the water valve 212 may be connected to a hot water source, a cold water source, or any combination thereof. A water line 216 may extend from the water valve 212 to a water coupler 218. An injector 220 may also be coupled to the water line 216 to inject cleaner into the water line 216. To this end, a container 222 of cleaner may be disposed outside of the cabinet 200 with an intake line 224 of the injector 220 feeding into the container 222, such that as the container 222 is depleted, a new container 222 of cleaner can replace it by simply moving the intake line 224 to the new container 222. The water valve 212 and injector 220 may be used to convey cleaning fluid (e.g., water, with or without cleaner) to the rover 102 through the water line 216 and water coupler 218 when the rover 102 is docked to the docking station 104.

The docking station 104 may also include a slidable cover 226 biased to be disposed over the waste coupler 208 and water coupler 218 to prevent debris from entering the waste line 206 and the water line 214 when a rover 102 is not docked to the docking station 104. When sufficient force is applied to the slidable cover 226, such as in a docking direction by a rover 102 being docked to the docking station 104, the slidable cover 226 may slide into the cabinet 200 so as to expose the waste coupler 208 and water coupler 218 for engagement with a draining circuit and cleaning circuit of the rover 102, respectively.

The docking station 104 may further include a pair of docking receivers 228 disposed on a front facing portion of the docking station 104. The rover 102 may have a corresponding pair of metal strike plates 230 disposed on a front facing portion of the rover 102. The docking receivers 228 may be configured to receive the strike plates 230 to mate the rover 102 with the docking station 104 during docking. It will be appreciated that the strike plates 230 and the docking receivers 228 may be reversed. In some implementations, the docking receivers 228 may be electromagnetically operable to magnetically adhere to the strike plates 230 when proximate each other.

The docking station 104 may additionally include a docking station controller 128B. Similar to the rover controller 128A, the docking station controller 128B may be configured to manage the components of the docking station 104, such as in accordance with instructions from the rover controller 128A when the rover 102 docks with the docking station 104. More particularly, the docking station controller 128B may be operatively coupled to the off-load pump 204, the water valve 212, and injector 220 to receive data from and/or control each of these devices. Similar to the rover controller 128A, the docking station controller 128B may include a processor 130B, a memory 132B, and a communications device 134B, each of which may be configured similarly to those of the rover controller 128A described above. In other words, the processor 130B may be configured to implement the functions, features, and processes of the docking station controller 128B described herein, such as by executing software embodied by computer-readable instructions residing in the memory 132B. The memory 132B may also store data accessed by the docking station controller 128B, or more particularly read by the processor 130B, to facilitate performance of such functions, features, and processes of the docking station controller 128B described herein.

The communications device 134B of the docking station controller 128B may provide a machine interface that facilitates data communication between the docking station controller 128B and one or more external systems and devices. For instance, responsive to a rover 102 being docked to the docking station 104, the communications device 134B of the docking station controller 128B may be configured to establish a connection, such as via a relatively short-range and/or direct connection wireless communications protocol, with the communications device 134A of the rover controller 128A so as to form a data connection between the docking station controller 128B and the rover controller 128A. In one example, the communications device 134B may be coupled to an IR transceiver 232 that is arranged on the docking station 104 such that, when the rover 102 is docked to the docking station 104, the IR transceiver 232 and the IR transceiver 136 of the rover 102 are within a line of site of each other. The rover controller 128A may then with the docking station controller 128B via the IR transceivers 136, 232, such as to select and initiate a certain type of cleaning cycle of the rover 102, exchange operational data of the rover 102, and/or exchange notification data as described herein.

In some implementations, the docking station 104 may be configured to function as an edge device for the rover 102 when docked with the docking station 104, such as via a data connection established with a remote processing system over one or more networks, including the Internet in some implementations. Thus, responsive to receiving operational data from the rover controller 128A via the data connection formed between the communication devices 134A, 134B, the docking station controller 128B may be configured to communicate the received operational data to the remote processing system for processing in the context of operational data received from other rovers 102 utilized by a health care system, and/or to determine whether a maintenance of usage related notification is appropriate for the rover 102. The communications device 134B of the docking station controller 128B may be configured to establish such data connection using relatively long range communications technologies such as Ethernet, fiber, cellular or Wi-Fi. As described above, unlike the communications device 134B of the docking station 104, the communications device 134A of the rover 102 may lack long-range communication capabilities and/or Internet access. In this way, transmissions between the rover controller 128A and devices external to the rover 102 may be limited when the rover 102 is being operated to collect medical waste during a procedure and/or not docked to the docking station 104, thus avoiding potential intraoperative disruptions to or changes in operation of the rover 102 caused by such data transmissions.

In some implementations, alternatively or in addition to the docking station 104, other devices in a health care facility with relatively long-range network connectivity, such as a medical hub or a mobile personal computing device (e.g., tablet), may be configured to function as edge devices for the rover 102 when the rover 102 is in proximity to the device. More specifically, when a rover 102 comes into communication range of such other device, the rover controller 128A may be configured to establish a wireless data connection with the device, such as using a relatively short range and/or direct connection wireless communication protocol, to enable the exchange of data therebetween. For example, in some implementations, a medical hub may be located outside of but nearby the docking station 104 such that when the rover 102 is docked to the docking station 104, the rover controller 128A forms a wireless data connection with the hub. In some examples, the docking station controller 128B may also maintain a data connection with hub, which may thus facilitate communication between the docking station 104 and the rover 102 when docked to the docking station 104, such as to trigger off-loading and cleaning cycles as described below.

Similar to the rover 102, the docking station 104 may include one or more sensors 234 in communication with the docking station controller 128B that are configured to generate operational data, or more particularly cleaning cycle data, for the rover 102 docked to the docking station 104. More specifically, the sensor(s) 234 may be configured to generate data indicative of characteristics of a draining cycle and/or cleaning cycle performed on the rover 102, including but not limited to at least one of water temperature data, water pressure data, detergent data, and cleaning cycle type data. For example and without limitation, the sensor(s) 234 may include a fluid measuring subsystem arranged to measure the volume of cleaning fluid supplied to the rover 102 during a cleaning cycle or drained from the rover 102 during a draining cycle. Additionally or alternatively, the sensor(s) 234 may include one or more flow rate sensors to measure a rate of flow of cleaning fluid passing into the rover 102 during a cleaning cycle and/or from the rover 102 during a draining cycle. Additionally or alternatively, the sensor(s) 234 may include one or more temperature sensors for measuring the temperature of cleaning fluid passing into the rover 102 during a cleaning cycle.

When the rover 102 is ready to be emptied, the rover 102 may be wheeled to the docking station 104 to mate therewith. The guide rails 202 on the docking station 104 may guide the rover 102 as it moves towards docking station 104 until the strike plates 230 engage the receivers 228. During such movement, the cart base 108 may cause the sliding cover 226 to retract into the cabinet 200 of the docking station 104, thereby exposing the docking station couplers 208, 218. Upon engagement of the strike plates 230 with the receivers 228, the docking station couplers 208, 218 may be aligned and mate with a set of corresponding couplers 236, 238 on the underside of the rover 102. More particularly, the docking station waste coupler 208 may couple to a corresponding waste coupler 236 of the rover 102 to allow waste material stored in the waste canister(s) 116 to be conveyed to the drain line 206 via the off-load pump 204. The docking station water coupler 218 may likewise couple to a corresponding water coupler 238 of the rover 102 to convey cleaning fluid into the waste canister(s) 116 of the rover 102 to clean the same.

Cleaning may be activated after the waste material has been off-loaded from the rover 102 to the drain line 206 during a draining cycle. To this end, the rover 102 may include draining circuit with an upper waste line 240 that extends between the upper waste canister 116A and the lower waste canister 116B for draining waste material from the upper waste canister 116A into the lower waste canister 116B. An upper waste valve 242 may be disposed in the upper waste line 240 and connected to the rover controller 128A to enable the flow of waste material from the upper waste canister 116A into the lower waste canister 116B.

The draining circuit may further include a lower waste line 244 that extends from the lower waste canister 116B to the rover waste coupler 236 for draining waste material in the lower waste canister 116B to the drain line 206 via the rover waste coupler 236 and docking station 104. More specifically, the rover waste coupler 236 may be a dry break coupling configured to prevent material from exiting the lower waste line 244 until coupled with the waste coupler 208 of the docking station 104, which may open the rover waste coupler 236 and thereby enable material to be pumped from the lower waste line 244 to the drain line 206 via the off-load pump 204.

Responsive to the rover 102 being docked to the docking station 104, a user may instruct the rover controller 128A to initiate a draining cycle to offload collected waste material, such as by interacting with a corresponding element of the user interface 138A of the rover 102. In response, the rover controller 128A may be configured to open the waste valve 242 to allow the waste material to drain from the upper waste canister 116A to the lower waste canister 116B, and may also trigger activation of the off-load pump 204, such as by communicating a corresponding instruction to the docking station controller 128B via the data connection formed between the rover controller 128A and the docking station controller 128B as described above. Responsive to receiving such instruction, the docking station controller 128B may be configured to operate the off-load pump 204 to pump the waste material out the lower waste line 244, through the rover waste coupler 236, the docking station waste coupler 208, and the waste line 210, and finally out to the drain line 206.

Following performance of the draining cycle, a cleaning cycle may be performed on the rover 102, such as in accordance with a user-selected type of cleaning cycle option. For instance, the user interface 138A may be configured to present varying cleaning cycle options for user selection, such as a “quick wash” cleaning cycle option, a “normal wash” cleaning cycle option, and an “extended wash” cleaning cycle option. The user's selection of a given type of cleaning cycle may be transmitted to the rover controller 128A, which may be configured to responsively trigger a cleaning cycle of the waste canister(s) 116 according to the selected type of cleaning cycle, such as by communicating a corresponding instruction to the docking station controller 128B via the data connection formed therebetween. The rover controller 128A may be configured to initiate the cleaning cycle of the waste canister(s) 116 automatically after the draining cycle has completed (e.g., after the waste canister(s) 116 have been emptied of waste material), which may be determined by the rover controller 128A based on data generated by the sensors 166 disposed in or adjacent the waste canister(s) 116, based on data generated by the sensors 234 of the docking station 104, and/or based on a runtime of the off-load pump 204.

Responsive to receiving the cleaning cycle instruction, the docking station controller 128B may be configured to open the water valve 209, and/or to inject cleaner from the container 222 into the water line 212 via the injector 220. The cleaning fluid may then flow through the docking station water coupler 218 and the rover water coupler 238 into a cleaning circuit of the rover 102, which may include a lower supply line 246 extending from the rover water coupler 238 to the lower waste canister 116B, and an upper supply line 248 extending from the lower supply line 246 to the upper waste canister 116A. A lower supply solenoid valve 250 may be disposed in the lower supply line 246 between the lower canister 116B and the upper supply line 248, and an upper supply solenoid valve 252 may be disposed in the upper supply line 248 between the upper waste canister 116A and the lower supply line 246. The lower supply solenoid valve 250 and upper supply solenoid valved 252 may be connected to and operable by the rover controller 128A.

Upon initiation of the cleaning cycle, the rover controller 128A may be configured to open the upper supply solenoid valve 252 to allow the cleaning fluid to flow under pressure from the lower supply line 246 to the upper supply line 248, and then to a sprinkler head disposed at the end of the upper supply line 248 for being sprayed about the interior of the upper waste canister 116A. Responsive to the cleaning fluid being sprayed in the upper waste canister 116A for a set period of spray time, which may correspond to the currently selected cleaning cycle option, the rover controller 128A may be configured to close the upper supply solenoid valve 252 and open the lower supply solenoid valve 250 to spray the cleaning fluid about the interior of the lower waste canister 116B. Responsive to the cleaning fluid being sprayed in the lower waste canister 116B for the set period of spray time, the rover controller 128A may then be configured to close the lower supply solenoid valve 250. Either during or after the waste canisters 116 are sprayed with cleaning fluid, the docking station controller 128B may be configured to operate the off-load pump 204 so as to dump the dirty cleaning fluid from the upper and lower waste canisters 116 into the drain line 206 as described above.

In the above-described example, the rover controller 128A may be configured to alternate between opening the lower supply solenoid valve 250 and upper supply solenoid valve 252 so as to alternate between spraying the lower and upper waste canister 116A, 116B. However, in other implementations, such as when sufficient water pressure is present, the rover controller 128A may be configured to open the supply solenoid valves 250, 252 simultaneously so as to spray the waste canisters 116 at the same time.

A given cleaning cycle performed on a rover 102 by the docking station 104 may include one or more wash phases and one or more rinse phases. During each wash phase, a cleaner-containing solution (e.g., water with cleaner) may be supplied by the docking station 104 to the rover 102 to be sprayed within the waste canisters 116 for a set duration and thereafter drained as described above. During each rinse phase, one of which may follow each wash cycle, water without cleaner may be supplied by the docking station 104 to the rover 102 to be sprayed within the canisters 116 for a set duration and thereafter drained as described above.

Different types of cleaning cycles may be associated with different durations in which the waste canisters 116 are sprayed during each wash phase and/or rinse phase of the cleaning cycle, and/or may be associated with different numbers of wash phase(s) and/or rinse phase(s) performed during the cleaning cycle. For instance, when a “quick wash” cleaning cycle is performed, the above-described wash and rinse phases may each be performed a relatively small number of times (e.g., each phase occurs once) and/or for a relatively short duration. When the “normal wash” cleaning cycle is performed, the above-described wash and rinse phases may each be performed a larger number of times (e.g., each phase is repeated two or three times) and/or for a longer duration. Finally, when the “extended wash” cleaning cycle is performed, the above-described wash and rinse phases may each be performed a further larger number of times (e.g., each phase is repeated five or six times) and/or for a further longer duration.

During each wash and rinse phase, the rover controller 128A may be configured to maintain the waste valve 242 in an open state and to trigger operation the off-load pump 204 so at to enable the dirty cleaning fluid to be pumped to the drain line 206 contemptuously with the spraying of fluid. Alternatively, in some implementations, when cleaning fluid is being sprayed into a waste canister 116, the rover controller 128A may be configured to maintain the waste valve 242 in a closed state and/or disable operation the off-load pump 204 until occurrence of an event, such as completing spraying each waste canister for the set duration.

In some implementations, when the waste valve 242 is transitioned from the closed to open state and/or when the off-load pump 204 is transitioned to an operative state may be dependent on the type of cleaning cycle being performed. For example and without limitation, when the quick cleaning cycle or the normal cleaning cycle is being performed, the rover controller 128A may be configured to maintain the waste valve 242 in an open state and the off-load pump 204 in an operative state throughout the wash and rinse phases so as to drain fluid from each waste canister 116 contemporaneously with the waste canister(s) 116 being sprayed. Conversely, when the extended cleaning cycle is being performed, the rover controller 128A may be configured to keep the waste valve 242 in the closed state and/or the off-load pump 204 in a disabled state for a set soak period following the cessation of the spraying of the waste canister(s) 116 so as to soak each waste canister 116 for a time, which may help to remove further soil, grime, or waste material from the waste canister(s) 116. Upon expiration of the set soak period, the rover controller 128A may be configured to open the waste valve 242 and/or enable operation of the off-load pump 204 to enable the dirty fluid to be pumped to the drain line 206 using the off-load pump 204.

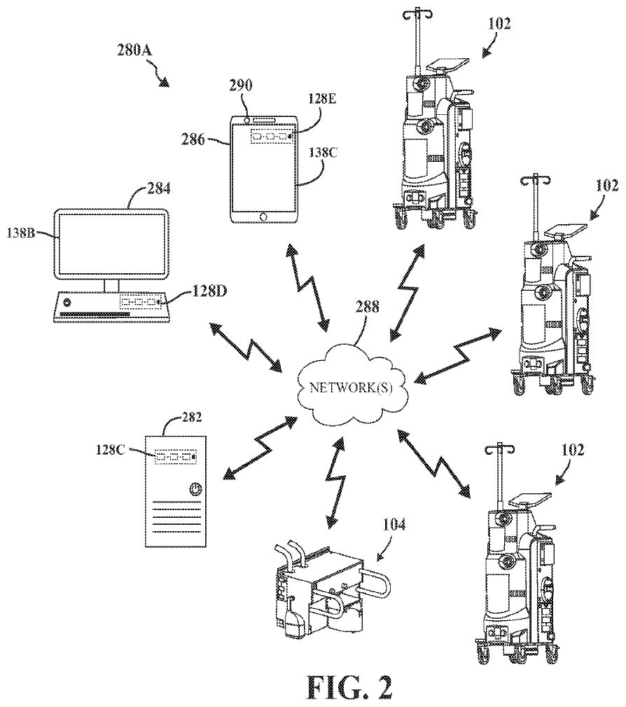

In some implementations, a user may be able to customize the parameters of a given cleaning cycle to be undergone by the rover 102. More particularly, responsive to the rover 102 being docked to the docking station 104, the rover controller 128A may be configured to provide a GUI on a display of the user interface 138A that enables selection of one of the available cleaning cycle options for an upcoming cleaning cycle, and also provides one or more user selectable elements for customizing parameters of the upcoming cleaning cycle For example and without limitation, the one or more selectable elements may enable the user to set cleaning cycle parameters such as a set spray duration, fill level, canister soak time, detergent volume, detergent addition timing, water pressure, water temperature, ingredient addition, or ultraviolet light activation. Additionally and/or alternatively, one or more of these parameters may be set using a remote user terminal 284 or mobile user device 286 (FIG. 2) in communication with the docking station 104 or rover 102, such as over one or more computer networks. In any event, responsive to receiving input that configures one or more of the above-described cleaning cycle parameters, the controllers 128A, 128B may be configured to perform a cleaning cycle on the docked rover 102 in accordance with the input.

As described above, a given healthcare facility or system may employ a fleet of rovers 102, each with varying usage and locations. FIG. 2 illustrates an example of a rover management and notification system 280A that may be implemented for facilitating optimal maintenance and operation of the rovers 102 in a manner that was previously unfeasible. As shown in the illustrated example, the rover management and notification system 280A may include a plurality of rovers 102, at least one docking station 104 for performing draining and cleaning cycles on the rovers 102, at least one remote server 282, at least one remote user terminal 284, and at least one mobile user device 286. Each of these components may be able to communicate with the other components over one or more computer networks 288. To this end, unlike the example described above, the rovers 102 in this example may include relatively long range communication capabilities so as to establish a data connection with the remote server(s) 282 over the computer network(s) 288, without for example establishing a relatively short range data connection with a docking station 104 or nearby mobile user device 286. The computer network(s) 288 may incorporate wireless and/or wired technologies, and may include, without limitation, one or more local area networks (LAN), metropolitan area networks (MAN), and/or wide area networks (WAN) such as the Internet.

In general, as the rovers 102 are used to collect medical waste during surgical procedures and are drained and cleaned in cooperation with the docking station(s) 104, the rovers 102 and/or docking station(s) 104 may be configured to communicate operational data related to such activity to the server(s) 284, such as over the one or more computer networks 288. The server(s) 282, which may generally form a processing system remote from the rovers 102 and/or docking station(s) 104, and may include one or more cloud servers and/or dedicated servers, may host one or more applications configured to aggregate the operational data from the different rovers 102, and track metrics for determining whether to trigger an action for the rover 102, such as the display of maintenance or usage notification as described below. The user terminal(s) 284 may be configured to provide user access to the rover management and notification system 280A, or more particularly to the applications hosted by the server(s) 282, such as to view data specific to each rover 102, including operational data, status data, and the tracked metrics associated with each rover 102, to view aggregate operational data of the fleet as a whole, and to receive notifications for maintaining the rovers 102. For example and without limitation, the user terminal(s) 284 may include one or more personal computing devices that are generally remote from the rovers 102, such as a desktop, laptop, or thin client terminal. The mobile user device(s) 286, which may include transient battery-powered personal computer devices such as a laptop, tablet or smartphone, may likewise function to access the rover management and notification system 280A, or more particularly to the applications hosted by the server(s) 282, as described herein. Additional details of the operation of the components of the rover management and notification system 280A are provided below.

The server(s) 282, user terminal(s) 284, and mobile device(s) 286 may include controllers 128C, 128D, 128E configured to implement the functions, features, and processes of the component described herein. Similar to the controllers 128A, 128B of the rovers 102 and docking station(s) 104 discussed above, each controller 128C, 128D, 128E may include a processor, memory, and communications device, which may be configured similarly to those of at least the docking station controller 128B described above. The user terminal(s) 284 and mobile device(s) 286 may also include respective user interfaces 138B, 138C for presenting data and notifications generated by the rover management and notification system 280A, and for receiving user input for regulating the same. For example and without limitation, each user interface 138B, 138C may include any one or more of the components of the user interface 138A described above in connection with each rover 102.

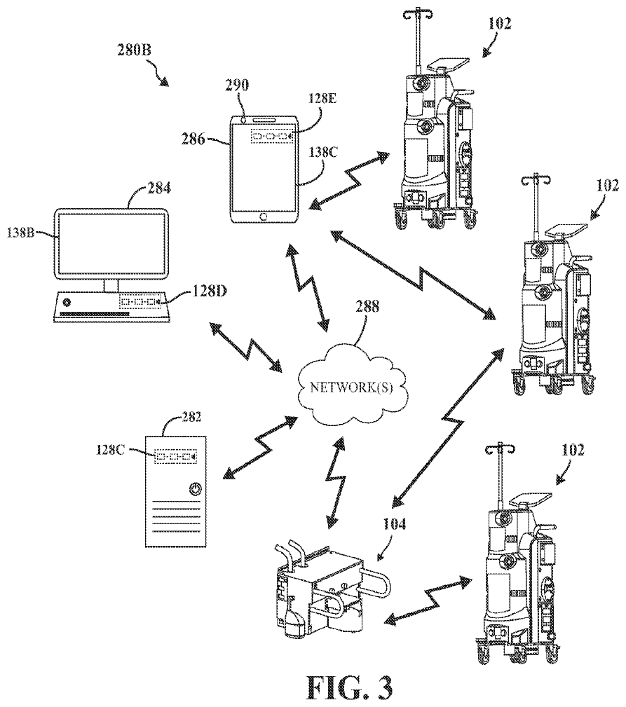

FIG. 3 illustrates an alternative example of a rover management and notification system 280B in accordance with the present disclosure. As discussed above, in some implementations, the communication capabilities of the rovers 102 may be relatively limited. For instance, the communication capabilities of the rovers 102 may be limited to relatively short-range and/or direct connection communication technologies, such as proximity-based wireless communication technologies (e.g., RFID, NFC), Bluetooth, Wi-Fi Direct, Zigbee or line-of-site based wireless communication technologies (e.g., IR). To this end, the rovers 102 may be unable to establish data connections with the server(s) 284 via the computer network(s) 288 directly. In this case, and as illustrated in the exemplary rover management and notification system 280B of FIG. 3, one of the other components of the rover management and notification system 280B, such as the docking station(s) 104 and/or mobile device(s) 286, may be configured to operate as an edge communication device for the rovers 102.

For instance and as described above, each rover 102 may be configured to establish a data connection, or more particularly a wireless data connection, with a docking station 104 when coupled to the docking station 104 for draining and/or cleaning using a relatively short-range communication technology such as IR. The docking station 104 may be configured with relatively long-range communications technology such as Ethernet, cellular, or Wi-Fi so as to facilitate communication between the docked rover 102 and the server(s) 282 over the computer network(s) 288. Additionally or alternatively, each rover 102 may be configured to establish a wireless data connection with a nearby mobile device 286 using a relatively short-range communication technology such as Bluetooth or Wi-Fi Direct. The mobile device 286 may be configured with relatively long-range communications technology such as Ethernet, cellular, or Wi-Fi so as to facilitate communication between the rover 102 and the server(s) 282 over the computer network(s) 288.

During operation of the rover management and notification system 280A, 280B, operational data for a given rover 102 of the fleet that is indicative of updated activity of the rover 102, including cleaning cycle data and/or surgical procedure data, may be sent to the server(s) 284 to be aggregated with similar data from the other rovers 102, which may facilitate providing an administrator with a complete view of the fleet of rovers 102 for planning future usage and replacement of components of the rovers 102, to check compliance with relevant policies (e.g., smoke evacuation policies, cleaning policies), and to determine a return on investment of the fleet of rovers 102. These tasks have conventionally not been possible or have been possible only to a very limited extent, such as by comparing order history to current inventory, conducting after-the fact interviews of clinical users, and attempting to compare historical and current red bag waste costs for a given department.

To this end, the rover management and notification system 280A, 280B may provide an application by which administrators and other non-clinical users may view aggregate data relating to the above matters (e.g., replacement components, compliance, return on investment) in different contexts, such as per rover 102, day, week, month, department, or type of rover 102. As an example, based on aggregate operational data generated from the operational data of individual the rovers 102, the rover management and notification system 280A, 280B may provide a graphical user interface, such as on a user interface 138B, 138C of a remote terminal 284 and/or the mobile device 286, that allows a user to view graphs of filter usage rates in the various contexts, including with and without smoke being present, graphs of surgical waste fluid disposal volume within various contexts, and add factors such as canister or red bag waste cost per liter of waste fluid to determine return on investment.

As a further example, the aggregation function of the rover management and notification systems 280A, 280B may enable a user to view graphs of manifold usage rates in various contexts so as to plan for future usage and ordering. For instance, during a given medical procedure involving a rover 102, the rover controller 128A of the rover 102 may be configured to generate manifold usage data by assigning a manifold use profile to each usage of a replaceable manifold 124 inserted in the rover 102. The use profile assigned to each usage of a replaceable manifold 124 may include one or more datums selected from the group consisting of date of use, time of use, medical specialty, type of procedure, associated rover type and/or model and/or unique identifier, and manifold type and/or part number and/or unique identifier. Additionally or alternatively, the rover controller 128A may be configured to generate manifold usage data by determining, with fluid measuring systems associated with the manifolds 124 (e.g., rover sensors 166), a fluid disposal volume for each usage.

The rover controller 128A of each rover 102 may be configured to transmit the manifold usage data, including the manifold use profiles and/or fluid disposal volumes, to the server(s) 284, which may be configured to generate and provide aggregate usage data for display, such as on a user interface 138B, 138C of a remote terminal 284 and/or mobile device 286, that is indicative of manifold usage characteristics across the fleet of rovers 102. As an example, the server(s) 284 may be configured to generate aggregate data at least in part by determining a fluid disposal cost based on the fluid disposal volumes and a weight-based disposal cost conversion, comparing the fluid disposal cost and an operational cost of the rovers 102, and displaying a cost savings on the user terminal 284, such as in reply to receiving a corresponding request from the user terminal 284. This analysis enabled by the aggregation of operational data from multiple rovers 102 may assist a user to quickly and accurately determine a cost benefit of the fleet of rovers 102 managed by the rover management and notification system 280A, 280B.

The aggregation function of the rover management and notification system 280A, 280B may also assist in the diagnosing of common issues with the rovers 102 on a fleet-wide basis. For example, each rover controller 128A may be configured to generate error codes based on abnormalities identified during operation of the rover 102. Error code data indicative of such error codes may be transmitted from each rover 102 to the server(s) 284, which in turn may generate aggregate error code data that is indicative of diagnostic patterns across the fleet of rovers 102. For example and without limitation, the transmitted error code data may include one or more datums selected from the group consisting of date of use, time of use, medical specialty, type of procedure, associated rover type and/or model and/or unique identifier, and part number. Transmission of such data to the server(s) 284 may also enable a technician to view, such as via a user terminal 284, a report of rover history including error codes with time/date stamps prior to dispatching service, which may help improve diagnostic and corrective action efficiency, saving time and money and minimizing rover downtime.

The server(s) 282 may be configured to provide the aggregate data for display on one of the user interfaces 138 of the other devices of the rover management and notification system 280A, 280B, such as the user terminal(s) 284, mobile device(s) 286, or rover(s) 102, using push communications. Alternatively, the aggregate data may be pulled from the server(s) 282 to a given one of the above devices periodically and/or on demand, such as based on user input received via the user interface 138 of the device. In some examples, responsive to a given rover 102 being coupled to a docking station 104, and optionally to corresponding user input being received on the user interface 138A of the rover 102, the docking station 104 may be configured to request the aggregate data from the server(s) 282 for display on the user interface 138A of the rover 102, such as via the docking station 104 acting as an edge device as described above.

In some implementations, a given mobile device 286 may be configured to function as a remote control for the rover 102, such as to trigger and control operation of the rover 102 to collect medical waste during a surgical procedure, via a data connection established between the mobile device controller 128E and the rover controller 128A when proximate to one another. Additionally or alternatively, such mobile device 286 may be configured to relay status information about the rover 102 to the server(s) 282 during a surgical procedure, such as when the rover 102 is currently applying suction to a surgical site, and operational data of the rover 102 in connection with the surgical procedure as described herein.

In some implementations, the mobile device 286 may further include a camera 290 or other scanner in communication with the mobile device controller 128E, which may be configured to operate the camera 290 to scan an ID badge of a practitioner before enabling the rover 102 to operate. To this end, the mobile device controller 128E may be configured to correlate the scanning of the ID badge with the starting of the procedure, and responsively communicate a corresponding status update indicative that that the rover 102 has entered a clinical state to the server(s) 282. The mobile device controller 128E may be configured to correlate a subsequent scanning of the badge with an end of the procedure, and responsively communicate a further status update to the server(s) 282 indicative of the same, along with operational data of the rover 102 generating during the procedure. In some implementations, the mobile device controller 128E may be configured to limit communications to and/or from the rover 102 when in the clinical state or applying suction, such as to avoid disruption to the rover's 102 operation.

In some implementations, upon recognizing the badge of a given practitioner, the mobile device controller 128E may be configured to set operational parameters of the rover 102 based on presets associated with the practitioner (e.g., suction levels, maximum flow rates). Additionally or alternatively, the mobile device controller 128E may be configured to display an interface for selecting the given type of procedure being performed with the rover 102, and to set operational parameters of the rover 102 based on presets associated with the selected procedure type. Further responsive to receiving a selected procedure type, the mobile device controller 128E may be configured to present instructions for use of the rover 102 relative to the specific to the type of procedure.

As described above, the operational data for a given rover 102 may include surgical procedure data indicative of operating characteristics of the rover 102 during one or more surgical procedures, which may be used by the rover controller 128A of the given rover 102, and/or by the server(s) 282, to determine whether to trigger an action relative to the given rover 102. At least a portion of such operational data may be generated by the sensors 166 of the rover 102, and may be transmitted to the server(s) 282 upon completion of each surgical procedure, such as via a connected mobile device 286, or upon being docked to the docking station 104. Additionally or alternatively, in some implementations, a mobile device 286 may also be used to generate operational data for a given rover 102, such as by being configured to image the waste canister(s) 116 with the camera 190 during and/or following a procedure, and/or to analyze the images to determine characteristics of the waste collection by the rover 102 in relation to the surgical procedure.

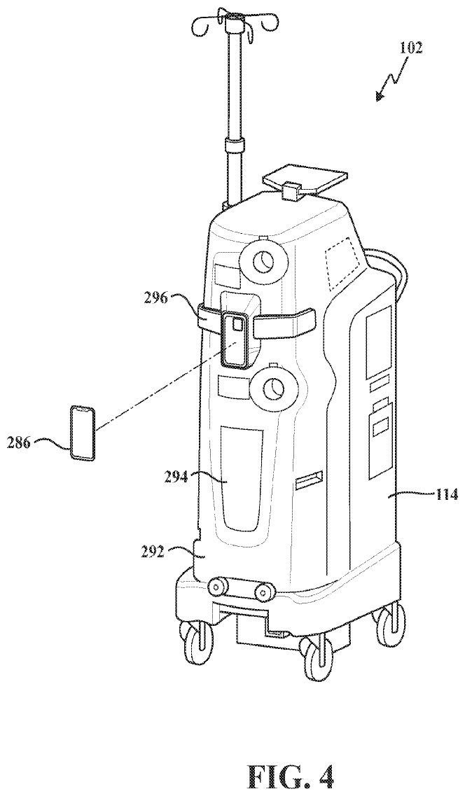

For instance, as illustrated in FIG. 4, a given rover 102 may include a front casing 292 coupled to the vertical chases 114 and that defines at least one cutout or window 294 to expose a portion of the waste canister(s) 116. The waste canister(s) 116 may be formed with transparent material through which a user may visually observe the waste material collected within the waste canister(s) 116, and, if needed, visually approximate a volume of the waste material collected therein with volumetric markings disposed on an outer surface of the waste canister(s) 116. The waste canister(s) 116 being optically clear also permits the waste material collected therein to be imaged by the camera 290 incorporated in the mobile device 286. The images from the camera 290 may be transmitted to and processed by controller 128E of the mobile device 286 to determine operational data of the rover 102 associated with a surgical procedure, such as at least one of a degree of occlusion of the collected waste material, a composition of the collected waste material (e.g., blood concentration), and a duration in which waste material is present in the waste canister(s) 116. More particularly, optical properties of the waste material may be analyzed and processed to determine such data, such as disclosed in commonly-owned U.S. Pat. No. 8,792,693, issued Jul. 29, 2014, and PCT Patent Application No. PCT/US2023/025603, filed Jun. 16, 2023, the entire contents of each of which are hereby incorporated herein by reference.

At least one device cradle 296 may be removably coupled or rigidly secured to the rover 102. The device cradle 296 may position the camera 290 relative to the waste canister(s) 116 in a precise manner to provide for continuous image capture (e.g., a video feed) of at least nearly an entirety of the waste canister(s) 116. The video feed results in continuous data from which the above properties may be determined in real-time, which as described in greater detail below, may be used to dynamically adjust one or more action notification thresholds, such as a cleaning notification threshold, for the rover 102 and thereby facilitate optimal performance of the same.

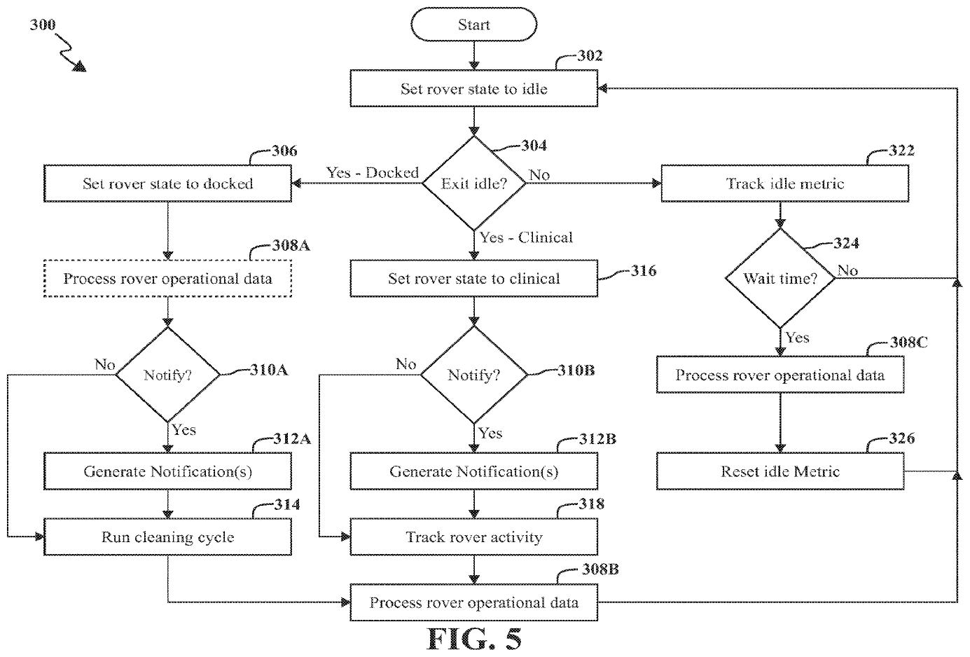

FIG. 5 illustrates a method 300 for dynamically managing the use and maintenance of a fleet of rovers 102 based on an analysis of the activity of the rovers 102, which may both prolong the useful life of the rovers 102 and promote operational efficiency of the fleet. The method 300 may be implemented by the rover management and notification system 280A, 280B, or more particularly by one or more controllers 128 of the rover management and notification system 280A, 280B, and may be performed relative to each rover 102 of the fleet.

In block 302, a state of the rover 102 may be set to idle. In general, when a rover 102 is made available to health care professionals for collecting medical waste, the rover 102 may initially enter an idle state, which may correspond to the rover 102 being inactive, powered off, in a low-power or standby mode, without a manifold 124 received in a receiver 120 of the rover 102, and/or not enabled to activate suction. The rover controller 128A and/or server controller(s) 128C may be configured to track a current state of each rover 102. In this way, responsive to a user checking a status of a given rover 102, such as via a user terminal 284, mobile device 286, or the user interface 138A of another rover 102, the rover controller 102 and/or the server controller(s) 128C may provide status data to the requesting device, such as over the computer network(s) 288, that indicates the current state of the rover 102 for display.

In block 304, a determination may be made of whether the rover 102 exits the idle state and enters a different state, such as a docked state or a clinical state. The docked state may generally correspond to the rover 102 being successfully docked to a docking station 104. The determination of whether the rover 102 is in the docked state may be made by the rover 102, or more particularly by rover controller 128A of the rover 102, or by the docking station 104, or more particularly by the docking station controller 128B of the rover docking station 104. In one example, a successful docking may be determined using sensors incorporated in the receivers 228 or strike plates 230. Alternatively, a successful docking may be detected based on communication being established between the rover 102 and docking station 104, such as via the communication devices 134A, 134B. For example, the docking station 104, or more particularly the docking station controller 128B, may be configured to determine a successful docking has occurred with a given rover 102 responsive to receiving communications from the rover controller 128A of the rover 102 via the IR transceivers 127, 229. Such communications may identify the specific rover 102 coupled to the docking station 104, such as by providing a unique identifier of the rover 102. Responsive to the rover 102 successfully docking with the docking station 104, the rover controller 128A may be configured to display a notification on the user interface 138A of the rover 102 indicative of the successful docking.

Responsive to determining that the rover 102 is in the docked state (“Yes—Docked” branch of block 304), in block 306, a status of the rover 102 may be set to the docked state, such as by the rover controller 128A and/or the server(s) 284. For instance, responsive to the rover 102 being docked, a status update may be communicated to the server(s) 284, such as from the rover 102 or docking station 104 over the computer network(s) 288, indicative of the docked state of the rover 102. In this way, responsive to a user checking a status of the given rover 102, such as via a user terminal 284, mobile device 286, or the user interface 138A of another rover 102 in communication with the server(s) 284, the server(s) 284 may provide status data to the requesting device that indicates the docked state of the rover 102 for display. The status data may further indicate the particular docking station 104, and also the location of the docking station 104 within the healthcare facility or system, for display. Additionally or alternatively, the rover controller 128A may store the docked state of the rover 102 locally, and may also be configured to reply to requests from remote devices with status data indicative of the rover's 102 docked state, the particular docking station 104, and/or the location of the docking station 104 within the healthcare facility or system for display.

In some implementations, responsive to the rover 102 entering the docked state, the method 300 may proceed block 308A, in which operational data of the rover 102 is processed. As previously discussed, the server controllers 128C may be configured to track one or more metrics associated with each rover 102 for determining whether a notification related to the rover 102 should be triggered based on the operational data of the rover 102. As previously discussed, the rover 102 may have relatively limited communication capabilities, which may render the rover 102 unable to directly communicate with the computer network(s) 288, such as for exchanging operational data with the sever(s) 284. In some instances, the rover 102 may be configured to pair with a mobile device 286, which may then function as an edge device for receiving operational data from the rover 102 for transmission over the computer network(s) 288 to the server(s) 282, such as upon completion of a procedure, and/or for receiving data (e.g., notification data, software updates) from the server(s) 282 over the computer network(s) 288 for transmission to the rover 102. However, if the rover controller 128A has operational data not yet uploaded to the server(s) 282, such as because a mobile device 286 was not available to pair with the rover 102, or the rover 102 is not configured to cooperate with a mobile device 286 to access the computer network(s) 288, then the method 300 may proceed to proceed to block 308A to responsive to the rover 102 be docked. Exemplary details of block 308A are described in reference to FIG. 7 below.

In block 310A, a determination may be made of whether a notification should be displayed on the rover 102, such as by the rover controller 128A of the rover 102 and/or the server controller(s) 128C. For instance, responsive to receiving the status update indicating the docked state of the rover 102, the server controller(s) 128C may be configured to look up and/or generate notification data for the rover 102, which may indicate whether a notification has been triggered for the rover 102. More specifically, the notification data may indicate any outstanding notifications for the rover 102, which may have been previously triggered as described in more detail below. The notification data may also indicate the type of triggered notification, such as a maintenance related notification (e.g., cleaning, filter) or a usage related notification. The server controller(s) 128C may be configured to transmit such notification data over the one or more computer networks 288 to the rover 102, or to the docking station 104, which may be configured to transmit a corresponding communication to the rover controller 128A over the data connection formed therebetween that causes the rover controller 128A to determine whether a notification should be displayed. Alternatively, the determination of block 306 may be made by the rover controller 128A of the rover 102 based on notification data that was previously generated and/or locally stored by the rover controller 128A, as described in more detail below.

Responsive to determining that a notification should be displayed on the rover 102 (“yes” branch of block 310A), in block 312A, one or more notifications may be generated and displayed on the user interface 138A of the rover 102, such as by the rover controller 128A. The content of the displayed notification(s) may depend on the type of outstanding notifications indicated by the notification data. For instance, a cleaning related notification may be configured to direct a user to perform a specific type of cleaning cycle on the rover 102, such as an extended cleaning cycle. In this way, a user may be directed to perform the specific type of cleaning cycle upon docking the rover 102 to the docking station 104 and prior to performing another type of cleaning cycle. A usage related notification may direct a user to use another, less frequency used one of the fleet of rovers 102 for a next medical waste collection procedure, such as to provide a balanced use of the fleet as a whole. A filter-related notification may direct a user to replace a filter of the rover 102, such as the aerosol filter 160 and/or the particle filter 164 described above.

Responsive to generating one or more notifications (block 312A), or to determining that a notification should not be generated (“No” branch of block 310A), in block 314, a cleaning cycle and/or draining cycle may be performed on the rover 102. As an example, a user may select and initiate a draining cycle and/or cleaning cycle via the user interface 138A of the rover 102, which in turn may cause the rover controller 128A to communicate an instruction to the docking station controller 128B to begin running the draining cycle and/or selected cleaning cycle. The docking station 104 may then perform a draining cycle and/or cleaning cycle on the rover as described above.

In block 308B, responsive to the waste canister(s) 116 being drained and/or cleaned by the docking station 104 according to the selected cleaning cycle, operational data for the rover 102 may be processed, such as based on characteristics of the cleaning cycle. The rover management and notification system 280A, 280B may maintain operational data specific to each rover 102 that spans various operational aspects of the rover 102. The operational data for each rover 102 may be maintained locally by the rover 102, such as in the memory 132A of the rover 102. Additionally or alternatively, the operational data for each rover 102 may be centrally maintained at the server(s) 284, such as in the memory 132C. As examples and without limitation, the operational data stored for each rover 102 may include cleaning cycle data for the rover 102 and/or surgical procedure data for the rover 102. The cleaning cycle data may generally indicate information relating to draining and/or cleaning cycles performed on the rover 102 by a docking station 104, such as indicated by the data generated by the sensors 166, 234 during such cycles. The surgical procedure data for the rover 102 may indicate information relating to use of the rover 102 to collect medical waste during surgical procedures, such as error code data, fluid collection volume data, filter usage data, vacuum pump runtime data, manifold usage data, procedure type data, patient data, and/or vision data determined through imaging the waste canister(s) 116 as described above. In some instances, the rover management and notification system 280A, 280B may also maintain administrative data for each rover 102, such as return on investment data for and/or service data for the rover 102.

In general, processing the operational data for the rover 102 in block 308B may include analyzing the operational data specific to the given rover 102 to determine whether to trigger a maintenance notification, such as a cleaning related or filter related notification, for the rover 102. The operational data for a given rover 102 may also be combined in block 308B with the operational data for other rovers 102 in the fleet to provide a comprehensive understanding of the fleet as a whole, and to also determine whether to trigger a notification, such as a usage related notification, to promote efficient allocation of the rovers 102. Additional details of block 308B are described in more detail in reference to FIG. 7 below.

Responsive to the operational data for the rover 102 being processed in block 308B, the method 300 may return to block 302 so as to set the state of the rover 102 back to idle. For instance, assuming the rover 102 is currently in the docked state, the rover 102 may be undocked from the docking station 104. Responsive to the rover 102 exiting the docked state, the rover controller 128A or the docking station controller 128B may be configured to communicate a status update to the server(s) 284 indicative that the rover 102 is no longer docked to the docking station 104, and has returned to the idle state. The server controller(s) 128C may be configured to store such status as part of the operational data specific to the rover 102. Additionally or alternatively, the rover controller 128A may be configured to reflect the idle state of the rover 102 in locally stored operational data of the rover 102.

As previously described in reference to block 304, the rover 102 may also exit the idle state by being placed in a clinical use state, such as in preparation for a surgical procedure. This determination may be made by the rover 102, or more particularly by the rover controller 128A of the rover 102. As non-limiting examples, the rover controller 128A may be configured to determine that the rover 102 enters the clinical use state responsive to the rover 102 being powered on when not docked to a docking station 104, responsive to the rover 102 receiving user input via the user interface 138A indicative to awake from a lower powered or standby mode, or responsive to one or more manifolds 124 being installed in the rover 102. Additionally or alternatively, this determination may be made by a controller 128E of a mobile device 286 connected to the rover 102, such as responsive to receiving user input indicating the rover 102 is to be used in a surgical procedure, and/or scanning an ID badge associated with a practitioner.

Responsive to determining that the rover 102 is in the clinical use state (“Yes Clinical” branch of block 304), in block 316, a status of the rover 102 may be set to the clinical state, such as by the rover controller 128A and/or the server controller(s) 128C. For instance, a status update may be communicated to the server(s) 284, such as from the rover 102 or a mobile device 286 connected to the rover 102, indicative of the clinical state of the rover 102. In this way, responsive to a user checking a status of the given rover 102, such as via a user terminal 284, mobile device 286, or the user interface 138A of another rover 102 in communication with the server(s) 284, the server(s) 284 may provide status data to the requesting device that indicates the clinical status of the rover 102 for display. The status data may further indicate the location of the rover 102 within the healthcare facility or system, for display. Additionally or alternatively, the rover controller 128A may store status data indicative of the clinical state of the rover 102 locally, and may also be configured to reply to requests from remote devices with status data indicative of the rover's 102 clinical state and/or the location of the rover 102 within the healthcare facility or system for display.

In block 310B, a determination may be made of whether a notification should be displayed on the rover 102, such as by the rover controller 128A of the rover 102 and/or the server controller(s) 128C. Block 310B may include processes similar to block 310A described above. For instance, responsive to receiving the status update indicating the clinical state of the rover 102, the server controller(s) 128C may be configured to look up and/or generate notification data for the rover 102, which may indicate whether a notification has been previously triggered for the rover 102. More specifically, the notification data may indicate any outstanding notifications for the rover 102, which may have been previously triggered as described in more detail below. The notification data may also indicate the type of triggered notification, such as a cleaning related notification, a usage related notification, or a filter related notification. The server controller(s) 128C may be configured to transmit such notification data over the one or more computer networks 288 to the rover 102, which may be configured to determine whether to display a notification based on the received data. Alternatively, the determination of block 310B may be made by the rover controller 128A of the rover 102 based on notification data that was previously generated and/or locally stored by the rover controller 128A, as described in more detail below.

Responsive to determining that a notification should be displayed on the rover 102 (“yes” branch of block 310B), in block 312B, one or more notifications may be generated and displayed on the user interface 138A of the rover 102, such as by the rover controller 128A. Block 312B may include processes similar to block 312A discussed above.

Responsive to generating one or more notifications (block 312B), or to determining that a notification should not be generated (“No” branch of block 31B), in block 318, activity of the rover 102 may be tracked. The rover 102 activity tracked in block 310B may generally correspond to at least a portion of the operational data maintained for each rover 102 that is described herein. More particularly, the rover 102, or more particularly the rover controller 128A of the rover 102, may be configured to track operational data, or more particular surgical procedure data, of the rover 102 as the rover 102 is used and/or maintained, such as based on data generated by the sensor(s) 166 of the rover 102 and/or vision data generated using with a mobile device 286 as described above. Such data may be used to update one or more metrics tracked for the rover 102 to determine whether to trigger an action for the rover 102 such as a notification.

As an example, tracking rover 102 activity in block 318 may include tracking usage of a vacuum source (e.g., vacuum pump 118) of the rover 102 for suctioning medical waste to generate vacuum runtime data for the rover 102. For instance, in response to each instance in which a vacuum source of the rover 102 is operated to draw medical waste into at least one of the waste canister(s) 116, the rover controller 128A may be configured to index a counter or initiate a timer, such as until the vacuum source ceases to draw medical waste into the canisters 116. In some implementations, the rover controller 128A may be configured to determine when medical waste is being drawn into canisters 116 based on when the rover controller 128A is operating a vacuum pump 118 of the rover 102, and/or based on data from one or more sensors 166 associated with each of the waste canister(s) 116, such as a weight sensor or a volume sensor.

Additionally or alternatively, tracking rover 102 activity in block 324 may include tracking filter usage of the rover 102 to generate filter usage data for the rover 102. For instance, the rover controller 128A may be configured to index a counter or initiate a timer specific to the aerosol filter 160 of the rover 102 in response to each instance of the rover 102 being operated in which a vacuum source, such as the blower 158 of the aerosol evacuation system 150, draws fluid through the filter 160 and/or in which the aerosol sensor 162 indicates that one or more aerosols are present or present above a preset non-zero threshold, such as until the blower 158 ceases drawing fluid through the filter 160 and/or the aerosol sensor 162 indicates that no aerosols are present or are present above the preset threshold. Similarly, the rover controller 128A may be configured to index a counter or initiate a timer specific to the particle filter 164 in response to each instance of the rover 102 being operated in which a vacuum source, such as the vacuum pump 118, draws fluid into the waste canister(s) 116, such as until the vacuum pump 118 ceases drawing fluid through the particle filter 164.

In some instances, tracking filter usage of the rover 102 may also include assigning a filter use profile to each usage of each filter 160, 164 during a medical procedure. Each assigned filter use profile may include one or more datums selected from the group consisting of date of use, time of use, medical specialty, type of procedure, associated rover type, model, and/or unique identifier, and filter type, model, and/or unique identifier.

Additionally or alternatively, tracking filter usage may include determining whether a filter change event has occurred. As an example, each filter 160, 164 for use with a rover 102 may include or be associated with an RFID tag indicative of whether the filter 160, 164 is new or used. The rover controller 128A may be configured to read this data from the RFID tags of each of the currently attached filters 160, 164 to determine whether the filter 160, 164 is new or used. Responsive to the read data indicating a given filter 160, 164 is new, the rover controller 128A may be configured to determine that a filter change event has occurred, and update the status of the filter 160, 164 to used, such by locally recording a unique identifier of the filter 160, 164 that is present on the RFID tag, and/or by writing data indicating that the filter 160, 164 is now used to the RFID tag.

Additionally or alternatively, tracking rover 102 activity in block 308 may include tracking manifold usage to determine manifold usage data for the rover 102. For instance, the rover controller 128A may be configured to assign a manifold use profile to each usage of a replaceable manifold 124 during a medical procedure. The use profile assigned to each usage of a replaceable manifold 124 may include one or more datums selected from the group consisting of date of use, time of use, medical specialty, type of procedure, associated rover type, model, and/or unique identifier, and manifold type, model, and/or unique identifier.

Additionally or alternatively, tracking rover 102 activity in block 318 may include tracking fluid disposal volumes for generating fluid disposal volume data for the rover 102. For instance, the rover controller 128A may be configured to track fluid disposal volumes for the rover 102 based on the data generated by the sensors 166 of the rover 102.

Additionally or alternatively, tracking rover 102 activity in block 318 may include tracking error codes generated by the rover 102 to generate error code data for the rover 102. For instance, the rover controller 128A may be configured to generate error codes based on abnormalities identified during operation of the rover 102, such as based on data generated by the sensors 166.

Following block 318, the method 300 may move to block 308B, in which operational data for the rover 102 may be processed, such as based on the rover 102 activity tracked in block 324. As previously mentioned, processing the operational data for the rover 102 in block 314 may include analyzing one or more metrics corresponding to the operational data specific to the given rover 102 to determine whether to trigger a notification, and to provide a comprehensive understanding of the fleet as a whole. Additional details of block 308B are described in more detail below in reference to FIG. 7.

Responsive to the operational data for the rover 102 being processed in block 308B, the method 300 may return to block 302 so as to set the state of the rover 102 back to idle, as described above. For instance, assuming the rover 102 is currently in the clinical state, the rover 102 may be powered down or placed in a low-powered or standby state, all manifolds 124 may be removed from the rover 102, and/or an input may be provided to a mobile device 286 connected to the rover 102 that is indicative of the end of the procedure.

While the rover 102 is in the idle state, the rover management and notification system 280A, 280B may be configured to periodically process the rover 102 operational data to determine, as an example, whether a notification should be triggered. Thus, continuing again with block 304, in response to determining that the rover 102 has not exited the idle state (“No” branch of block 304), in block 322, an idle metric may be tracked. For instance, an idle counter may be indexed or an idle timer may be initiated (if not already initiated), such as by the rover controller 128A or the server controller(s) 128C. In block 324, a determination may be made of whether the idle metric has reached a preset value corresponding to a desired wait time. If so (“Yes” branch of block 324), then the method 300 may proceed to block 308C, in which operational data of the rover 102 may be processed. Block 308C may generally correspond to blocks 308A and 308B, as described below. Thereafter, in block 326, the idle metric may be reset.

In the above-described examples, the rover 102 is illustrated as entering the idle state from the clinical state and the docked state. However, as will be appreciated by those of ordinary skill in the art, in other implementations, the rover 102 may also transition from the docked state to the clinical state, and vice versa, without necessarily entering the idle state.

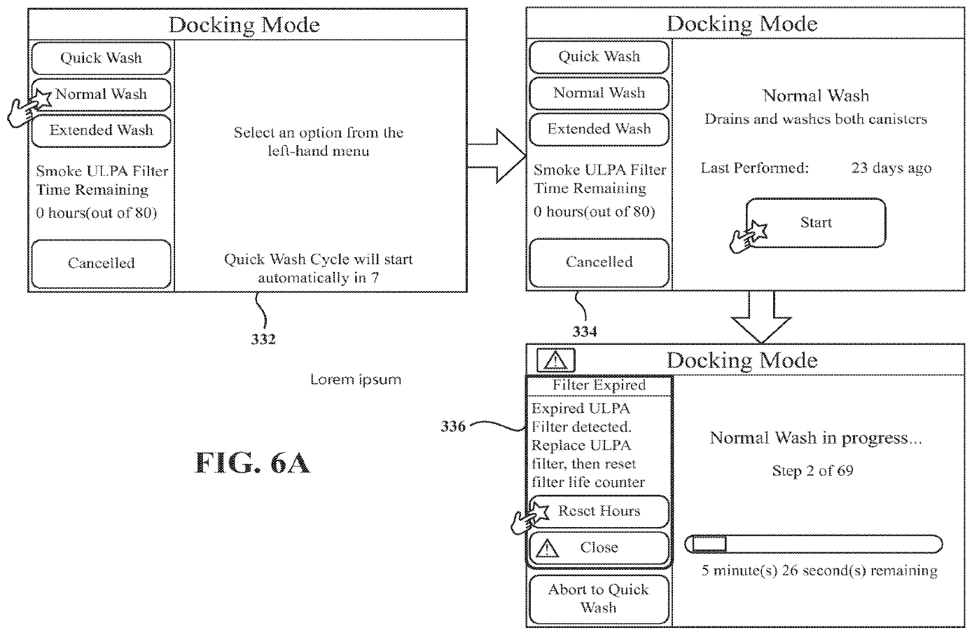

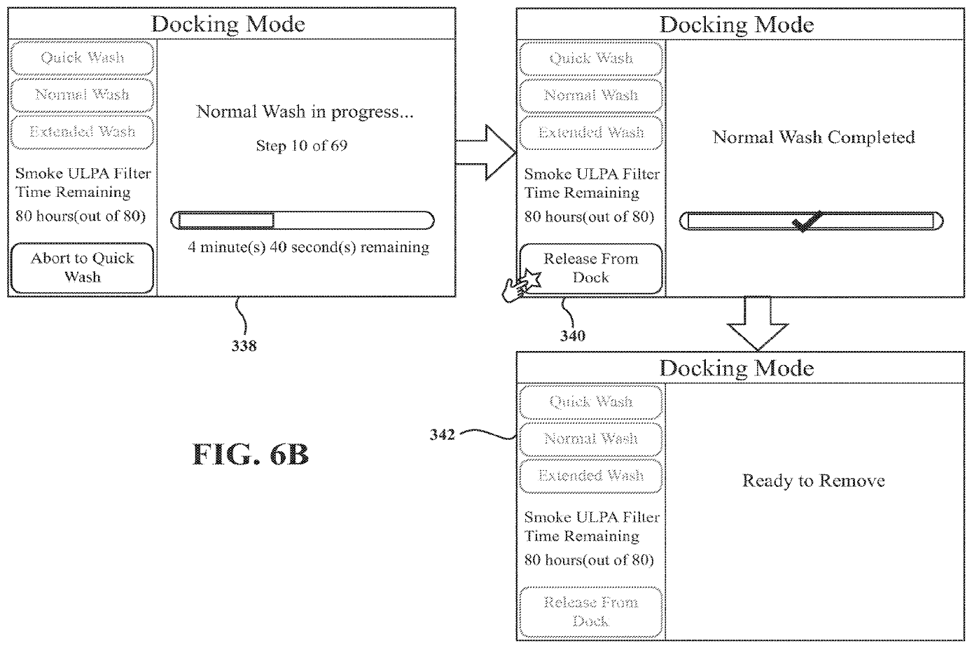

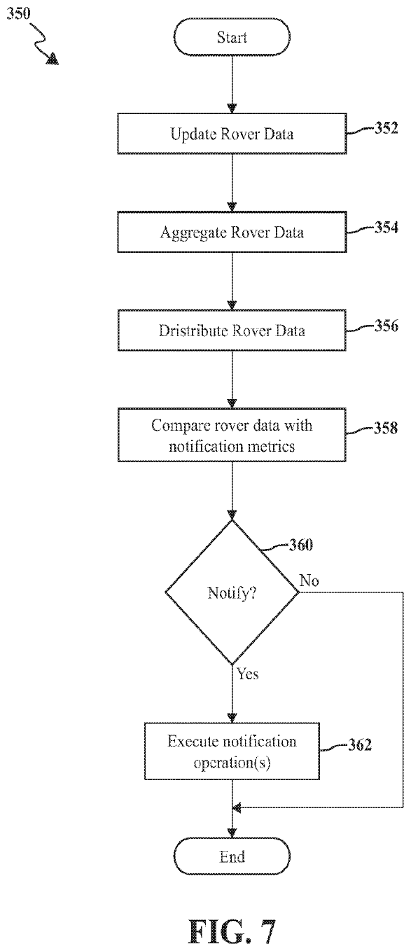

FIGS. 6A and 6B illustrates screens of a GUI that may be generated on the user interface 138A of the rover 102 when the rover 102 enters the dockets state, such as when block 310A indicates that a filter related notification should be triggered on the rover 102. Responsive to the rover 102 being docked, the GUI may show a screen 332 that includes varying types of selectable cleaning cycles, along with a filter notification metric indicating a remaining life of the filter 160. Responsive to selection of a given type of cleaning cycle, in this case the “normal wash” cleaning cycle, the GUI may proceed to screen 334 in which the user is presented with a selectable element to start performance of the selected cleaning cycle. Responsive to performance of the selected cleaning cycle being initiated, the GUI may illustrate a further screen 336 including a status bar indicating a progression of the cleaning cycle. The screen 336 may also provide one or more notifications determined for the rover 102, in this case a filter related notification that directs the user to replace the filter 160 and then reset the filter notification metric. Upon replacement of the filter 160 and selection of the reset element, the GUI may proceed to screen 338 to show the reset filter notification metric and also the cleaning cycle status bar. Upon completion of the cleaning cycle, the GUI may then proceed to the screen 340 to indicate that the cleaning cycle is completed, and to also provide a selectable element for releasing the rover 102 from the docking station 104. Finally, upon selection of the release element, the GUI may show the screen 342 providing an indication hat rover 102 is ready to be removed form the docking station 104.

FIG. 7 illustrates a method 350 for processing the operational data of the rover 102, such as to determine whether a notification should be triggered and/or to provide a comprehensive understanding of the fleet as a whole. The method 350 may be performed in blocks 308A, 308B, and/or 308C of the method 300 illustrated in FIG. 5, and may be implemented by the rover management and notification system 280A, 280B, or more particularly one or more controllers 128 of the rover management and notification system 280A, 280B.

In block 352, the operational data specific to the rover 102 may be updated, such as based on the cleaning and other activity of the rover 102 discussed above, and such as by the rover controller 128A and/or the server controller(s) 128C. As previously described, the rover management and notification system 280A, 280B may maintain operational data specific to each rover 102 that spans various operational aspects of the rover 102, and provides a comprehensive snapshot of the rover 102 to enable efficient utilization and maintenance of the rover 102 and the fleet as a whole. The operational data for each rover 102 may be maintained locally by the rover 102, such as in the memory 132A of the rover 102. Additionally or alternatively, the operational data for each rover 102 may be centrally maintained at the server(s) 284, such as in the memory 132C if server(s) 284. In this case, updating the operational data specific to the rover 102 in block 352 may include transmitting locally recorded operational data to the server(s) 284 over the network(s) 288, such as from the rover 102, and/or via the docking station 104 to which the rover 102 is docked, and/or via a mobile device 286 to which the rover 102 is paired. The locally recorded operational data may generally include data related to operation of the rover 102 that has not yet been transmitted to the server(s) 284. For example and without limitation, the transmitted data may include one or more of cleaning cycle data for the rover 102, error code data for the rover 102, filter data for the rover 102, vacuum pump runtime data for the rover 102, manifold usage data for the rover 102, and/or vision data for the rover 102.

More specifically and without limitation, datums that may be provided in the operational data for a given rover 102 may include: Rover part number; Rover serial number; Total number of rover power-on events, including mains power ups and docking power ups; Total rover power on time; Total number of vacuum pump start-up events; Total rover vacuum pump on time; Average vacuum pump current for all time; Average vacuum pump current before each of the last three cleaning cycles; Total number of smoke motor start-up events; Total smoke motor on time; Total ULPA filter usage; Total HEPA filter usage; Last time HEPA filter life was reset; Last time ULPA filter life was reset; Total number of times HEPA filter life has been reset; Total number of times ULPA filter life has been reset; Total number of completed quick wash cleaning cycles; Total number of attempted quick wash cleaning cycles; Last time quick wash cycle was completed; Total number of completed normal wash cleaning cycles; Total number of attempted normal wash cleaning cycles; Last time normal wash cycle was completed; Total number of completed extended wash cleaning cycles; Total number of attempted extended wash cleaning cycles; Last time extended wash cycle was completed; Maximum cleaning cycle flow rate experience by Rover; Sum of all cleaning cycle flow rates experienced by Rover; Minimum cleaning cycle flow rate experienced by the Rover; Total number of successful docking cycle flow rate calculations by the Rover; Maximum peak cleaning cycle water temperature experienced by Rover; Sum of maximum peak cleaning cycle water temperatures experienced by Rover; Minimum peak cleaning cycle water temperature experienced by the Rover; Sum of minimum peak cleaning cycle water temperatures experienced by Rover; Total number of successful cleaning cycle water temperature calculations by the Rover; Most recent cleaning cycle flow rate experienced by the Rover; Most recent peak cleaning cycle water temp experienced by the Rover; Total waste fluid collected and offloaded by the Rover; Total number of manifolds used by each canister 116 of the rover 102; Day of data download; Total number of attempted tank dumps; Total number of completed tank dumps; Total number of volume resets; Total time the Rover's IV pole UP button has been pressed; Date/time stamp of the last time Rover updated this data; Total number of 1-port manifolds used on this Rover; Total number of 4-port manifolds used on this Rover; Total number of 4-port SC manifolds used on this Rover; Total #of manifolds used on this Rover today; Total #of manifolds used on this Rover yesterday; Total #of manifolds used on this Rover 2 days ago; Total #of manifolds used on this Rover 3 days ago; Total #of manifolds used on this Rover 89 days ago; Most recent error code (left of decimal); Most recent error date/time stamp (UTC); 2nd most recent error code (left of decimal); 2nd most recent error date/time stamp (UTC); 200th most recent error code (left of decimal); 200th most recent error date/time stamp (UTC).

Additionally or alternatively, the operational data updated in block 352 may include notification metrics used to determine whether a notification for the rover 102 is appropriate. Correspondingly, updating the operational data specific to the rover 102 in block 352 may include updating one or more of these metrics, such as by the rover controller 128A and/or the server controller(s) 128C.

As one example, the operational data maintained for each rover 102 may include one or more cleaning related metrics specific to the rover 102. For instance, the cleaning related metric(s) may include a metric corresponding to a time since a last cleaning cycle, or since a first application of suction by the rover 102 since a last cleaning cycle. Additionally or alternatively, the cleaning related metric(s) may include a counter and/or a timer tracking usage of the rover 102 since a last cleaning cycle or since a first application of suction by the rover 102 since a last cleaning cycle, which similar to that of the vacuum pump runtime data discussed above may be indexed or initiated, such as by the rover controller 128A, responsive to each instance the rover 102 is operated to collect medical waste.

Responsive to completion of a cleaning cycle on the rover 102, the cleaning related metric(s) may be reset to zero in block 352. Assuming such metric(s) are maintained by the server(s) 284, responsive to completion of a cleaning cycle on a given rover 102, the rover 102, or the docking station 104 to which the rover 102 is docked, or the mobile device 286 to which the rover 102 is paired, may be configured to send a to the server(s) 284 over the computer network(s) 288 indicative of completing of the cleaning cycle, which may cause the server controller(s) 128C to reset the cleaning related metrics for the rover 102. In some implementations, at least one of the cleaning related metrics maintained for each rover 102 may be specific to the performance of a particular type of cleaning cycle, such as an extended wash cleaning cycle. Such cleaning related metric(s) may be reset responsive to the rover 102 being subject to an extended wash cleaning cycle, but not other cleaning cycle options.

A further cleaning related metric that may be updated in block 352 is a cleaning cycle ratio specific to the rover 102. The cleaning cycle ratio may be a ratio of the number of times the rover 102 is subject to a cleaning cycle according to one or more cleaning cycle options (e.g., quick wash cleaning cycle and/or standard wash cleaning cycle) to a number of times the rover 102 is subject to a cleaning cycle according to the extended wash cleaning cycle option.

Further as described above, updating the operational data specific to the rover 102 in block 352 may include updating one or more filter related metrics specific to the rover 102, which may include one or more filter metrics indicative of a time since each filter 160, 164 was last replaced, a time since suction was first provided by the rover 102 since the particle filter 164 was replaced, and/or a time since the aerosol evacuation unit 150 was first operated since the aerosol filter 164 was last replaced. Additional or alternatively, the filter related metrics may include at least one counter and/or timer tracking usage of each filter 160, 164, such as based on the data generated by the sensor 162 of the aerosol evaluation unit 150 (e.g., for the filter 160) and/or based on the vacuum runtime data (e.g., for the filter 164) discussed above. Responsive to a filter change event being determined, such as of the aerosol filter 160 or the particle filter 164, updating the operational data specific to the rover 102 may include resetting the filter metrics associated with the changed filter 160, 164 to zero.

Responsive to updating the rover specific data in block 352, the method 350 may proceed to block 354, in which aggregate rover data may be updated. More specifically, the rover-specific operational data may be combined with operational data from other rovers 102 to form aggregate rover operational data. In general, the aggregate rover operational data may provide information about each of the rovers 102 in the context of the fleet as a whole, so as to enable smart decision making relative to servicing and allocating the various rovers 102 to different surgical procedure. The aggregate rover operational data may also include one or more metrics for determining whether a notification is appropriate in the context of the fleet, as described in more detail below. In some implementations, the aggregate rover operational data may be updated by the server controller(s) 128C.

For instance, updating the aggregate rover operational data in block 354 may include determining relative cleaning data indicative of one or more relative cleaning related metrics specific to the fleet of rovers 102, such as based on the updated rover cleaning cycle data for the given rover 102 (and that of the other rovers 102 for which the method 300 is performed). More particularly, the server(s) 284 may be configured to consolidate the cleaning cycle data of each rover 102 of the fleet to generate one or more relative cleaning related metrics of the fleet, such as a ranked list of relative cleanliness of the fleet of rovers 102. For instance, the ranked list of relative cleanliness may be based on the value(s) of the cleaning-related metric(s) tracked for each of the rovers 102 of the fleet as described above.

Additionally or alternatively, updating the aggregate rover operational data in block 354 may include determining relative usage data of the fleet of rover 102. Unlike the rover specific usage data, such as indicated by the vacuum pump runtime data described above, which may include data specific to one rover 102 of the fleet, the relative usage data may include metrics indicative of the relative usage of two or more of the rovers 102. Such data may thus enable efficient utilization of the fleet across multiple locations to maximize resources, such as to avoid one rover 102 in the fleet being used in a majority of procedures while another rover 102 remains sparsely used.

The relative usage data may generally be generated by comparing the rover usage data specific to one rover 102 to the rover usage data specific to other rovers 102 of the fleet, the comparison being indicative of a relative usage of the rovers 102. For instance, the server controller(s) 128C may be configured to determine a relative usage metric for a given pair of rovers 102 by subtracting an operating duration indicated by received rover usage data, such as indicated by a counter or timer of the vacuum pump runtime data described above, associated with one of the rovers 102 of the pair from an operating duration indicated by received rover usage data associated with the other rover 102 of the pair, with the magnitude of the difference being utilized as a relative usage metric for the pair of rovers 102. Additionally or alternatively, the server controller(s) 128C may be configured to determine a relative usage metric for a given pair of rovers 102 by determining a usage ratio for the rovers 102, which may be a ratio of the operating duration indicated by the rover usage data for one of the rovers 102 of the pair to the operating duration indicated by the usage data for the other rover 102 of the pair. In some implementations, the server controller(s) 128C may be configured to generate a given relative usage metric for each possible combination of two or more rovers 102 of the fleet. As a further example, the relative rover usage data may include a ranked list of relative usage of the rovers 102, which may be generated, at least in part, according to relative usage metrics described above.

In block 358, the aggregate rover operational data may be distributed to one or more devices of the rover management and notification system 280A, 280B, such as each of the rovers 102, the user terminal(s) 284, and/or the mobile device(s) 286, for selective display on these devices. An exemplary GUI for illustrating such data on a given one of the above devices is described in more detail below.

In block 360, the rover specific operational data and/or the aggregate rover data, or more particularly the metrics indicated by such data, may be compared to one or more corresponding notification thresholds to determine whether to trigger any notifications. The rover specific operational data may be compared by the controller 128A of the given rover 102 or the server controller(s) 128C, while the relative rover operational data may be compared by the server controller(s) 128C. In some implementations, a user may be able to customize the thresholds, such as by interacting with a user interface 138 of the rover management and notification system 280A, 280B.

For instance, comparing the rover specific operational data in block 358 may include comparing the cleaning related data for the given rover 102 to corresponding preset thresholds. More particularly, the rover controller 128A and/or server controller(s) 128C may be configured to compare each cleaning related metrics against a corresponding threshold value. Additionally or alternatively, comparing the rover specific operational data in block 358 may include comparing the filter usage data for the given rover 102 to one or more preset thresholds. More particularly, the rover controller 128A or the server controller(s) 128C may be configured to compare each filter usage metric to a corresponding threshold value.

Additionally or alternatively, comparing the aggregate rover data in block 358 may include comparing the relative rover usage data with one or more corresponding thresholds. For instance, the server controller(s) 128C may be configured to compare each difference magnitude described above with a threshold difference, and/or may be configured to compare each relative usage ratio with a threshold usage ratio. As one non-limiting example, the threshold usage ratio may be at least 150%.

In some implementations, the rover controller 128A or the server controller(s) 128C may be configured to dynamically update a given notification threshold for the rover 102 based on the operational data for the rover 102. As an example, when a cleaning cycle, or a cleaning cycle of a particular type (e.g., an extending wash cleaning cycle), should be performed on a given rover 102 to promote optimal function of the rover 102 may depend on usage of the rover 102 indicated by the operational data. For instance, the greater the blood concentration, hold time, and/or occlusion level of the waste collected by the rover 102, such as indicated by the vision data, the sooner it may be desirable to perform a cleaning cycle, or more particularly an extended cleaning cycle, on the rover 102. Moreover, the lower the water pressure and/or temperature of the cleaning fluid provided by a facility when cleaning a given rover 102, the more often it may be desirable to perform a cleaning cycle, or more particularly an extended cleaning cycle, on the rover 102. The type of procedure and/or conditions of the patient may also factor into how often cleaning cycles, such as an extended cleaning cycle, should be performed. For instance, if a patient on which the rover 102 is used has a highly viral condition, it may be desirable to perform a cleaning cycle, or more particularly an extended wash cleaning cycle, on the rover 102 sooner than if not. The rover controller 128A or the server controller(s) 128C may be configured to factor in such information indicated by the operational data for the rover 102 to adjust (e.g., reduce) a cleaning related notification threshold accordingly.

As another example, the useful life of each filter 160, 164 may very as a function of its expected life and also the composition of fluid to which the filter 160, 164 is exposed, which may be indicated by the operational data of the rover 102, such as in the data generated by the aerosol sensor 162 and the vision data indicating characteristics of the collected medical waste respectively. The rover controller 128A or the server controller(s) 128C may thus be configured to adjust a filter notification threshold for a given rover 102 each filter metric based on an expected life of the corresponding filter 160, 164 and such operational data.

In block 360, based on the comparison(s), a determination may be made whether to trigger a notification. Such determination may be made by the device performing the comparison (e.g., the rover controller 128A, the server controller(s) 128C). In one implementation, responsive to one of the above comparisons indicating the compared metric (e.g., cleaning related metric, filter usage metric, relative usage metric) is greater than its corresponding preset threshold, a determination may be made that a notification should be triggered.

In general, determining that a notification should be triggered in block 360 may function to dynamically update a usage or maintenance schedule for the rover 102 or fleet of rovers 102, and generate a notification relating to the same. For instance, the rover controller 128A and/or the server controller(s) 128C may be configured to determine whether a cleaning schedule for a rover 102 should be dynamically updated and a notification relating to the same should be generated based on the comparison of the cleaning related data for the rover 102. Similarly, the rover controller 128A and/or server controller(s) 128C may be configured to determine whether a filter maintenance schedule for a given rover 102 should be dynamically updated and a notification relating to the same should be generated based on the comparison of the filter usage data for the rover 102. Likewise, the server controller(s) 128C may be configured to determine whether a usage schedule for the fleet of rovers 102 should be dynamically updated and a notification relating to the same should be generated based on the comparison of the relative usage data.

Responsive to determining that a notification should be triggered (“Yes” branch of block 360), in block 362, one or more notification operations may be executed, such as by the server controller(s) 128C and/or the rover controller 128A. In some implementations, such as when the server controller(s) 128C determine that a notification should be triggered, notification data corresponding to the comparison implicating the triggered notification may be generated for the given rover 102 at the server controller(s) 128C. In this way, responsive to the rover 102 later communicating with server(s) 284, such as in one or more blocks 308A, 308B and 308C described above, notification data may be forwarded to the rover 102 to cause the rover 102 to display a notification corresponding to the notification data.

Additionally or alternatively, executing one or more notification operations in block 362 may include generating and displaying a notification in real time relative to the determination that a notification triggered, such as on the user interface 138A of the given rover 102, and/or on the user interface 138B of a user terminal 284, and/or the user interface 138C of a mobile device 286 registered to the given rover 102. For instance, the given rover 102 and/or server(s) 284 may be configured to generate and push such notification to one or more of the above devices, such as based on stored contact data indicative of devices subscribed to the given rover 102 and their contact information (e.g., phone number, IP address, email address) associated with the given rover 102. In this way, a notification can be provided to the person best suited to receive such notification. For example, when the filter 160 of the aerosol evacuation system 150 or the particle filter 164 is due for replacement, the rover 102 could be configured to notify the clinical user by locally prompting them to replace it soon. However, clinical users are often not the same users that actually purchase and replace the filters. Accordingly, in addition or rather to displaying a filter-related notification on the rover 102, the server controller(s) 128C may cause a notification to be displayed on a user terminal 284 or mobile device 286 of the person best suited to receive such notification.

As previously described, the content of the notification may depend on the notification type, which in turn may depend on the comparison that implicated the notification triggering event. For instance, each comparison of relative usage data may involve rover usage data specific to at least two rovers 102. Responsive to a comparison of such data indicating that a notification should be triggered, the notification generated from this comparison may direct a user to use a less frequency used one of the rovers 102 implicated by the comparison, such as indicated by the rover usage data specific to the rovers 102 implicated by the comparison. As an example, for a given group of rovers 102 implicated by a comparison causing such a notification, the server controller(s) 128C may be configured to generate notification data for each rover 102 for which the relative rover usage data indicates more frequent use than a less frequently used rover 102 such that, when a given one of the more frequently used rovers 102 is docked or enters a clinical state, a notification is provided on the user interface 138A of the rover 102 that directs the user to use the less frequently used rover 102 of the group.

As a further example, responsive to the filter usage data for a given rover 102 causing a determination of a notification triggering event, executing notification operation(s) in block 362 may include triggering a notification on any user terminal 284 subscribed to the rover 102, such as the user terminal 284 of an administrator in charge of ordering and replacing rover filters 160, 164.

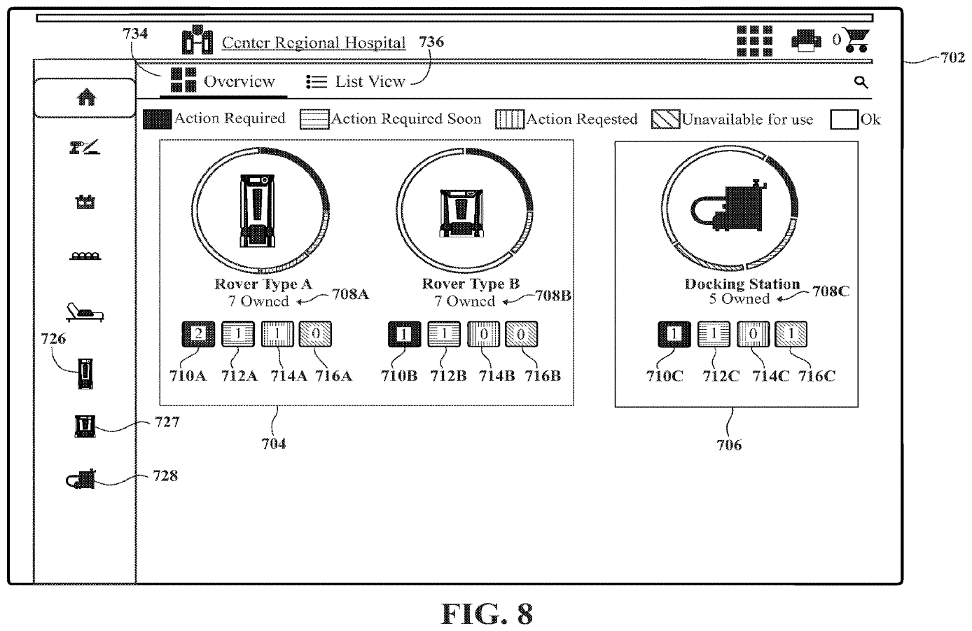

As previously described, the rover management and notification system 280A, 280B may provide an application by which administrators and other non-clinical users may obtain a complete view of the fleet of rovers 102 for planning for future usage and replacement of components of the rovers 102, to check compliance with relevant policies (e.g., smoke evacuation policy), and determine a return on investment of the fleet of rovers 102. FIG. 8 illustrates a home screen 702 of a GUI that may be generated on the user interface 138A of the rovers 102, and/or the user interface 138B of the user terminal 284, and/or on the user interface 138C of a mobile device 286, such as in accordance with the above exemplary methods. As shown in the illustrated example, the home screen 702 may include a rover summary portion 704 and a docking station summary portion 706, each providing various datums about the stock of corresponding devices deployed by a healthcare facility or system.

The rover summary portion 704 may include at least one datum indicating a number of rovers 102 deployed by the given healthcare facility or system. In some instances, the healthcare facility or system may utilize rovers of various types. For example, a given healthcare facility or system may employ one or more type A rovers 102 and one or more type B rovers 102. Relative to a type A rover 102, a type B rover 102 may have different features, such as by being smaller and more portable, but able to hold less waste. In this case, the rover summary portion 704 may include a rover type A rover datum 708A indicative of the number of type A rovers 102 owned by the healthcare facility or system, and may include a type B rover datum 708B indicative of the number of type B rovers 102 owned by the healthcare facility or system.

For each rover datum 708A, 708B, the rover summary portion 704 may also indicate a status of the stock of the rovers 102 of the given type. As an example, for each rover datum 708A, 708B, the rover summary portion 704 may include an action datum 710A, 710B indicative of the number of rovers 102 of the given type currently needing action (e.g., maintenance), an upcoming action datum 712A, 712B indicative of a number of rovers 102 of the given type needing action soon, an action requested datum 714A, 714B indicative of the number of rovers 102 of the given type for which action has been requested, and an unavailability datum 716A, 716B indicative of the number of rovers 102 of the given type that are currently unavailable for use (e.g., not functional).

Similarly, the docking station summary portion 706 may include a docking station datum 708C indicative of a number of dockings stations 104 owned by the healthcare facility or system, an action datum 710C indicative of the number of docking stations 104 currently needing action (e.g., maintenance), an upcoming action datum 712C indicative of a number of docking stations 104 needing action soon, an action requested datum 714C indicative of the number of docking stations 104 for which action has been requested, and an unavailability datum 716C indicative of the number of docking stations 104 that are currently unavailable for use (e.g., not functional).

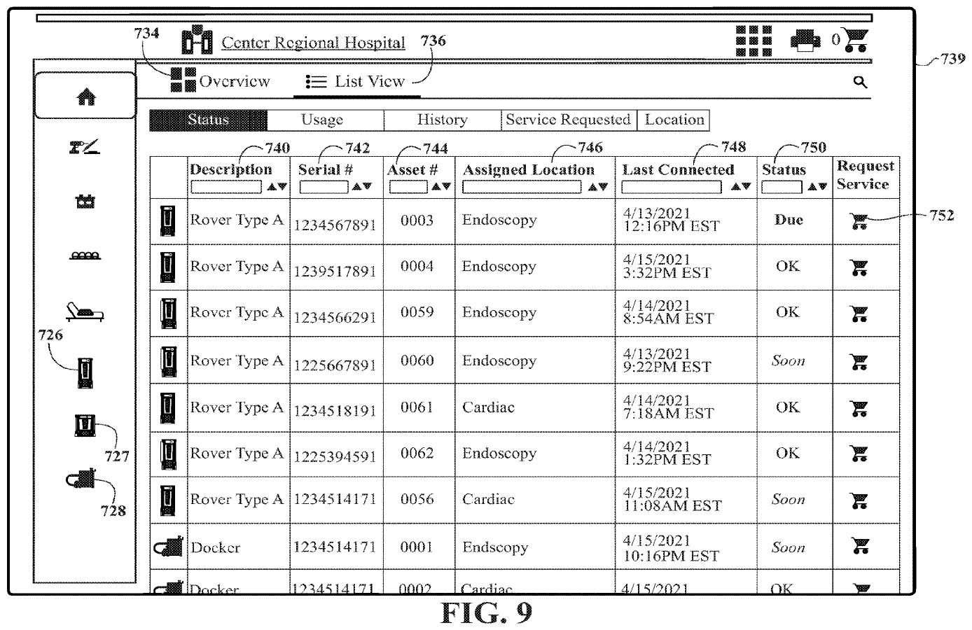

FIG. 9 illustrates another screen 739 that may be generated by the GUI, such as in response to user selection of the list view option 736 in the screen 730 (FIG. 8). The screen 739 may present a detailed view of the rovers 102 and docking stations 104, such as in the form a table with a row for each rover 102 or docking station 104 owned by the healthcare facility or system. The table may also provide various datums for each rover 102 or docking station 104, including, but not limited to, one or more of a description datum 740, a serial number datum 742, an asset number datum 744, an assigned location datum 746, a last connected datum 748, which may indicate the last time the rover 102 was docked to a docking station 104 or paired to a mobile device 286, and a status datum 750, which may indicate a current service need of the rover 102 or docking station 104 (e.g., whether the rover 102 or docking station 104 currently needs service or is expected to need service soon).

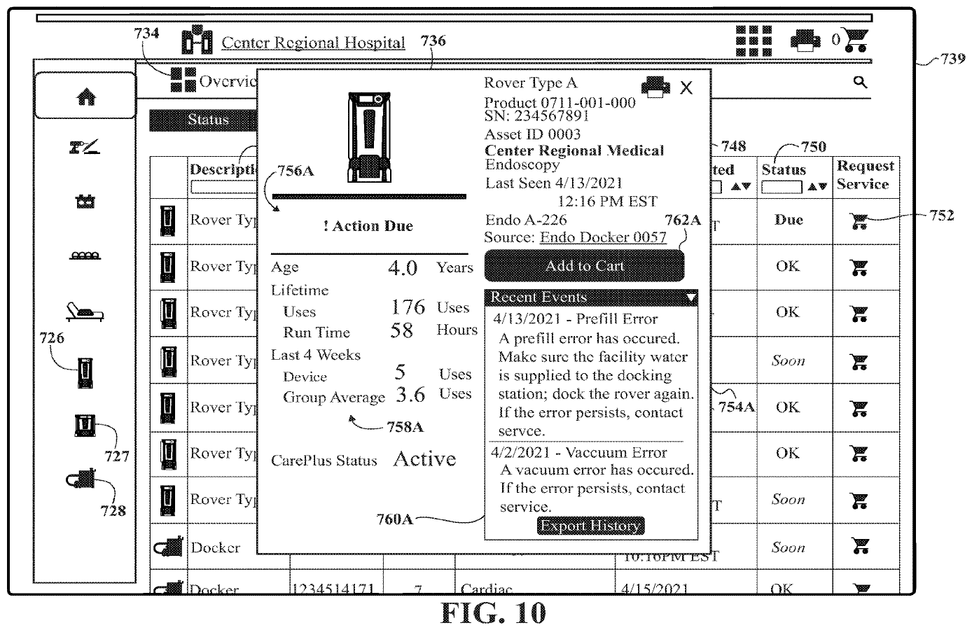

As shown in the illustrated example, each rover 102 and docking station 104 may also be associated with a user-interactive service request element 752 for requesting service for the rover 102 or docking station 104. FIG. 10 illustrates a service request window 754A that may be generated by the GUI responsive to a user selection of the service request element 752 associated with a rover 102 indicated as needing service. As shown in the illustrated example, the service request window 754A may include an informative portion 756A indicative that service is needed for the rover 102. The service request window 754A may also include a usage history portion 758 indicative of historical usage of the rover 102, including a relative usage datum indicative of the average number of rover 102 uses in the last four weeks, and a recent event portion 760 indicative of recent events of the rover 102, such as recent errors and/or services. In this way, when the request is forwarded to the service team, it is accompanied with a history of events and usage information that provides some context for the service team to address the issue needing services.

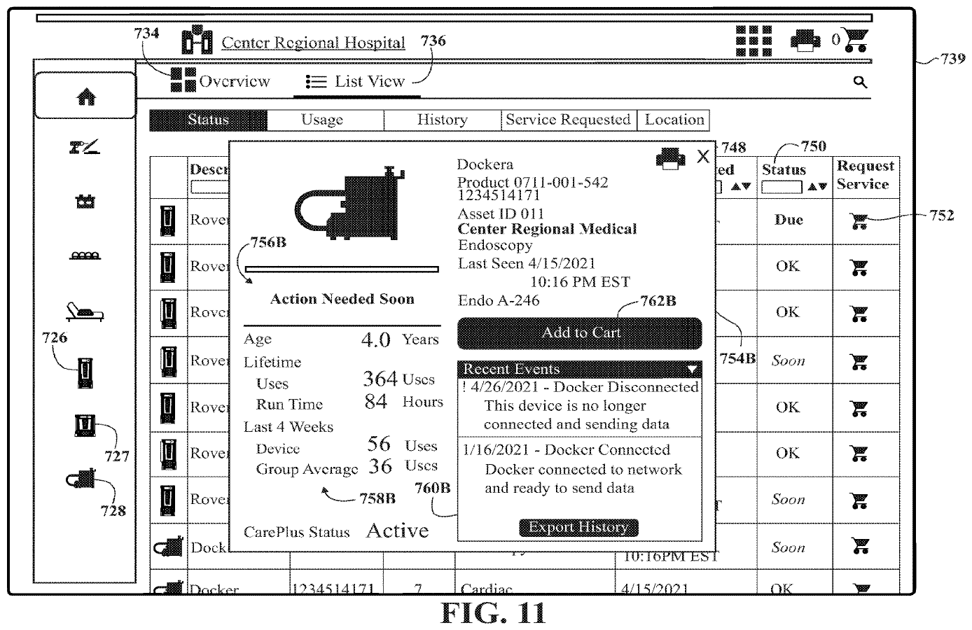

Finally, the service request window 754A may also include a user-selectable “Add to Cart” element 762A, which may be selected by the user to place the service request in a cart of the user. The cart may generally serve as a temporary hold for each service request desired by the user. Specifically, once a user has determined and added each desired service request to the cart, the user may then navigate to the cart by selecting a user-interactive cart element 752, and submitting each of the service requests from the cart at the same time. FIG. 11 illustrates a service request window 754B that may be generated by the GUI response to a user selection of the service request element 752 associated with a given docking station 104, and includes similar information and components as the service request window 754A.

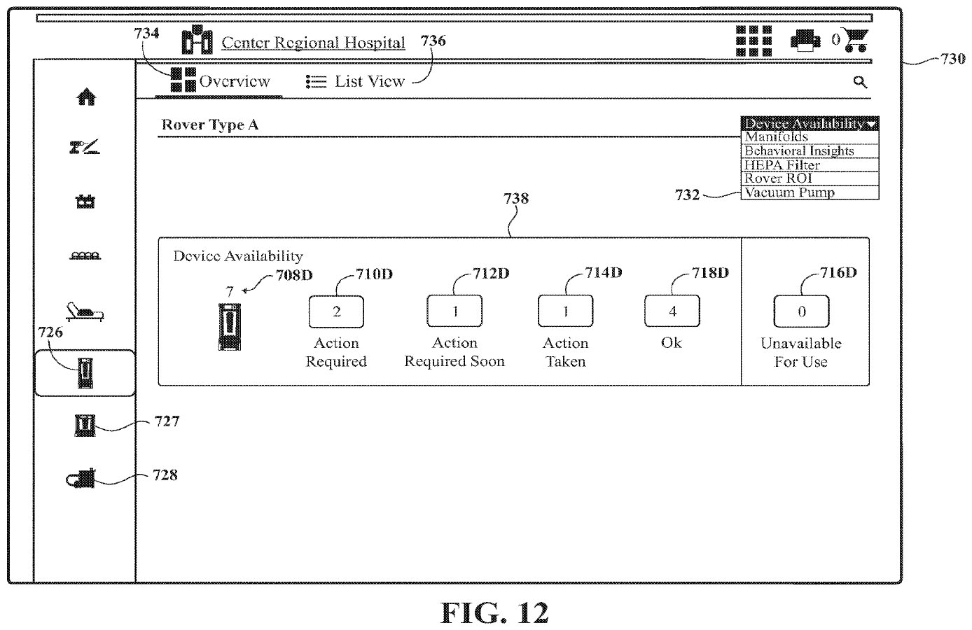

FIG. 12 illustrates a screen 730 that may be shown by the GUI responsive to a user selection of the user-selectable element 726 corresponding to the type A rover module. Although the foregoing figures describes exemplary screens relative to the type A rover 102, it will be understood that the GUI may be configured to show similar screens responsive to selection of the user-selectable element 727 corresponding to the type B rover 102, but with data specific to the type B rover 102.

As shown in the example illustrated in FIG. 12, the screen 730 may include a user-interactive navigational element 732, such as in the form of a drop-down list, for navigating between the different views of aggregate data for the type A rover 102. In the illustrated example, “Device Availability” is currently selected from the navigational element 732.

The screen 730 may further include a rover summary portion 738, which may include information similar to the information about the type A rovers 102 provided in the rover summary portion 704 of the home screen 702 (FIG. 8). To this end, the rover summary portion 738 may include a rover type A rover datum 708D indicative of the number of type A rovers 102 owned by the healthcare facility or system, may include an action datum 710D indicative of the number of type A rovers 102 currently needing action (e.g., maintenance), an upcoming action datum 712D indicative of a number of type A rovers 102 needing action soon, an action requested datum 714D indicative of the number of type A rovers 102 for which action has been requested, and an unavailability datum 716D indicative of the number of type A rovers 102 hat are currently unavailable for use (e.g., not functional). The rover summary portion 738 may also include an available rover datum 718D indicative of the number of type A rovers 102 that are available for use and are neither in need of an action nor in need of action soon.

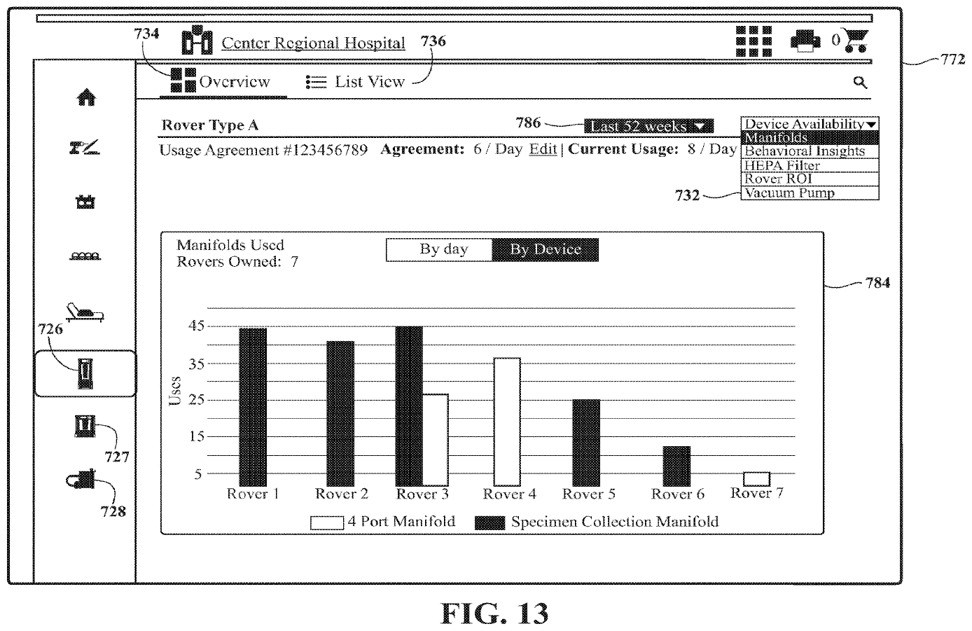

FIGS. 13 to 17 illustrate further screens 772, 774, 776, 778, 780, 782 that may be generated by the GUI responsive to the user selecting other views from the navigational element 732 while in the type A rover module corresponding to the user-selectable element 726. In general, each of the screens 772, 774, 776, 778, 780, 782 illustrated in these figures may present a different aspect of the aggregate data described above so as to provide a comprehensive understanding of the operation of the fleet of rovers 102. As shown in the illustrated examples, each of the screens 772, 774, 776, 778, 780, 782 may include the user-interactive navigational element 732 for navigating between the different screens 772, 774, 776, 778, 780, 782 to view different aspects of the aggregate data.

The screen 772 illustrated in FIG. 13 may correspond to selection of “Manifolds” in the navigational element 732, and may generally be configured to display aggregate manifold usage data across the fleet of rovers 102. To this end, the screen 772 may include a graph 784 illustrating the number of each of one or more types of manifolds used by each of the rovers 102 over a given period, which may set using a user-interactive period selector 786.

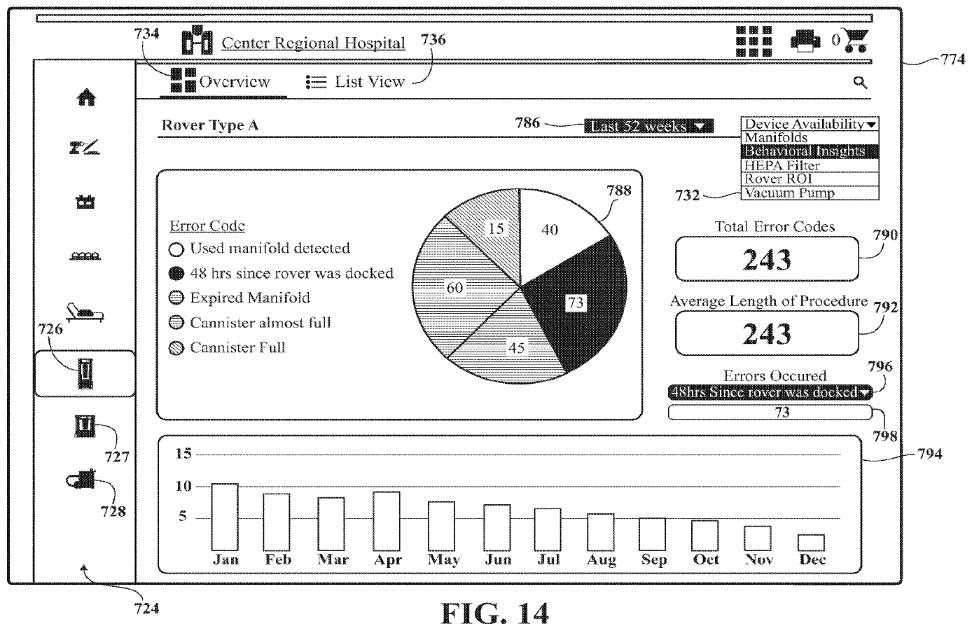

The screen 774 illustrated in FIG. 14 may correspond to selection of “Behavioral Insights” in the navigational element 732, and may generally be configured to display aggregate error code data across the fleet of rovers 102. To this end, the screen 774 may include a pie chart 788 indicating the number and types of errors generated across the fleet of rovers 102 over a given period, which may set using the user-interactive period selector 786. The screen 774 may also include a total errors datum 790 indicative of a total number of recorded errors over the given period, and may include an average procedure length datum 792 indicative of the average length of a procedure in which the type A rovers 102 were used over the given period, which may be determined based on the duration in which the rovers 102 were operating to collect waste.

The screen 774 may further include a bar graph 794 indicative of the number of a given type of error that occurred during each of defined sub periods (e.g., months) of the given period (e.g., fifty two weeks). The type of error represented by the bar graph 794 may be selected via a user-interactive error type selector 796, which may be in the form of a drop down menu. The screen 774 may also include a field 798 associated with the error type selector 796 such that, responsive to a given error type being selected vie the error type selector 796, the field 798 may be updated to indicate the number of errors of the selected type that occurred over the given period.

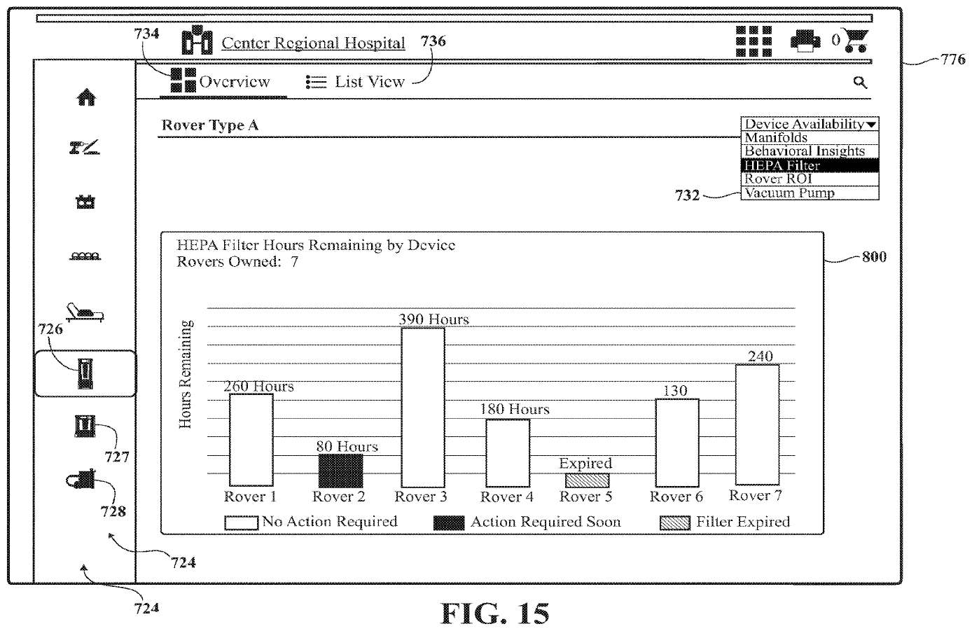

The screen 776 illustrated in FIG. 15 may correspond to selection of “HEPA Filter” in the navigational element 732, and may generally be configured to display aggregate filter use data across the type A rovers 102. To this end, the screen 776 may include a bar chart 800 indicating a remaining life of the particle filter 164 of each type A rover 102, and/or indicating whether the particle filter 164 of each rover 102 is in need of replacement or is expected to be in need of replacement soon. A similar screen may be provided by the GUI relative to the aerosol filter 160.

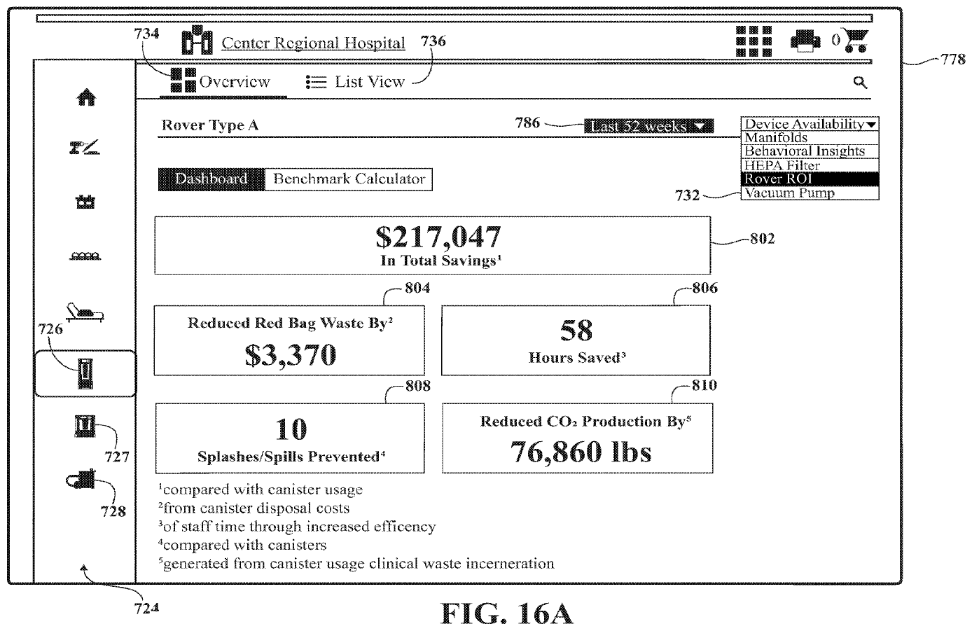

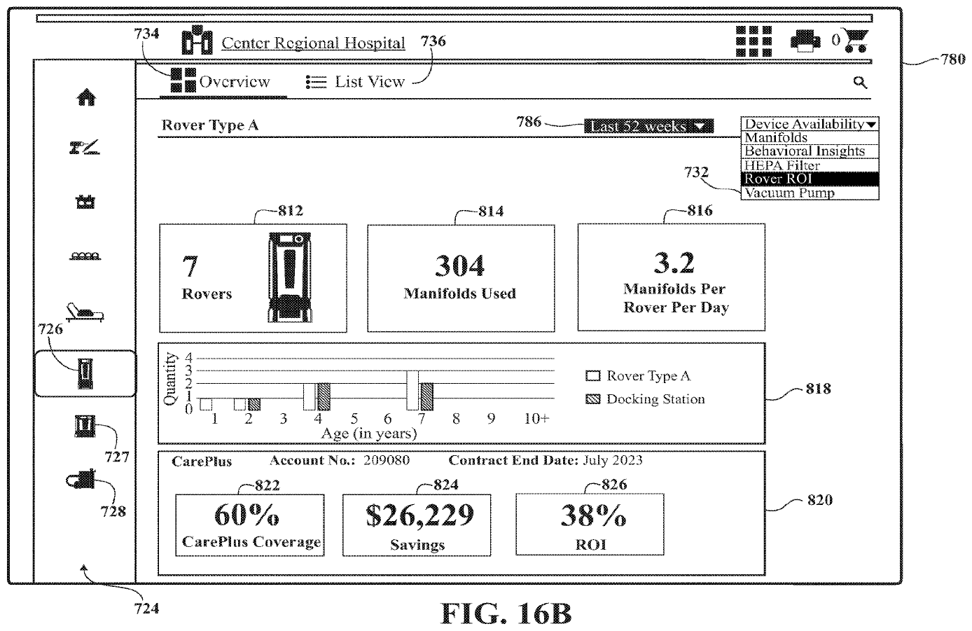

The screen 778 illustrated in FIG. 16A may correspond to selection of “Rover ROI” in the navigational element 732, and may generally be configured to display aggregate return on investment data across the fleet of type A rovers 102. To this end, the screen 778 may include several datums calculated by the server(s) 284 based on the operational data received from the type A rovers 102 that indicate the return on investment being obtained by the type A rovers 102. Such datums may include, without limitation, a total savings datum 802 indicative of a total amount of savings provided by the type A rovers 102 relative to alternative waste collection and disposal methods, a red bag savings datum 804 indicative of savings in disposal of red bag waste relative to alternative waste collection and disposal methods, an hours saved datum 806 indicative of a number of hours of staff time saved through increased efficiency relative to other waste collection and disposal methods, a contamination prevention datum 808 indicative of a number splashes/spills prevented relative to other waste collection and disposal methods, and a carbon impact datum 810 indicative of a reduced amount of CO₂ production relative to other waste collection and disposal methods.

The screen 780 illustrated in FIG. 16B may also correspond to the selection of “Rover ROI” in the navigational element 732, and may generally be configured to display further aggregate data relating to a return on investment of the fleet of type A rovers 102. To this end, the screen 778 may include several datums, at least some of which may be calculated by the server(s) 284 based on the operational data received from the type A rovers 102 that relate to an ROI for the type A rovers 102. For instance, the displayed datums may include, without limitation, a rover number datum 812 indicative of the number type A rovers 102 in service by the healthcare facility or system; a total manifolds datum 814 indicative of a number of manifolds 124 that may have been used over a given period, which may be set using the user-interactive period selector 786; and a daily manifolds datum 816 indicative of an average number of manifolds 124 used per day over the given period. The screen 780 may also include a bar chart 818 indicative the age of the type A rovers 102 and/or the docking stations 104 to which the type A rovers 102 may be docked, and may include a protection plan summary section 820 indicative of protection plan coverage for the type A rovers 102. The protection plan summary section 820 may include one or more datums relating to protection plan coverage for the type A rovers 102 such as, without limitation, a percentage coverage datum 822 indicative a percentage of the type A rovers 102 covered by the protection plan, a protection plan savings datum 824 indicative of a savings provided by the protection plan, and a ROI datum 826 indicative of a return on investment of the protection plan.

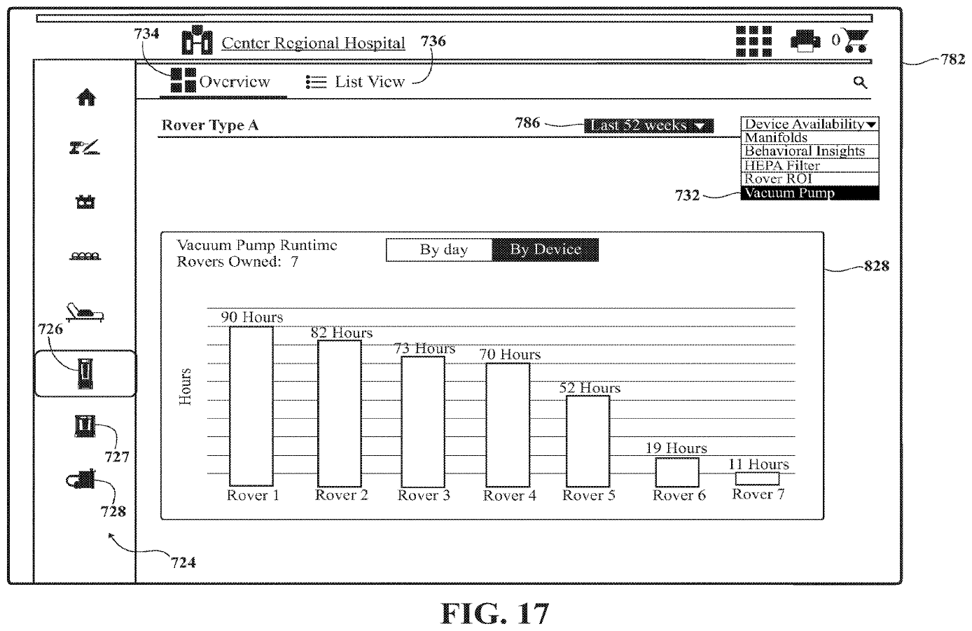

The screen 782 illustrated in FIG. 17 may correspond to selection of “Vacuum Pump” in the navigational element 732, and may generally be configured to display vacuum pump runtime data across the fleet of type A rovers 102. To this end, the screen 782 may include a bar chart 828 indicating a total runtime of each vacuum pump 113 of each of the type A rovers 102 over a given period, which may be set using the user-interactive period selector 786.

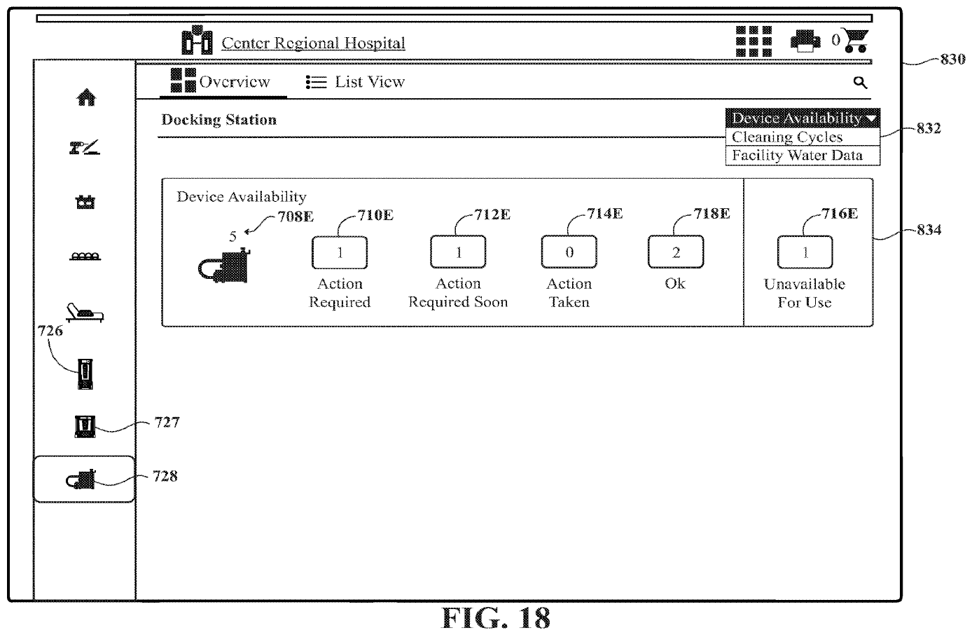

It will be appreciated that the GUI may likewise be configured to show similar screens showing aggregate data across the docking stations 104 of the rover management and notification system 280A 280B, such as when the user-interactive element 728 corresponding to the docking station module is selected. FIG. 18 illustrates a further screen 830 that may be shown by the GUI upon selection of the user-interactive element 728 corresponding to the docking station module.

The screen 830 may include a user-interactive navigational element 832, such as in the form of a drop-down list, for navigating between the different views of aggregate data for the docking stations 104. In the illustrated example, “Device Availability” is currently selected from the navigational element 832. Corresponding to this selection, the screen 830 may further include a docking station availability summary portion 834, which may include information similar to the information about the docking stations 104 provided in the docking station summary portion 706 of the home screen 702 (FIG. 8). To this end, the docking station availability summary portion 834 may include a docking station datum 708E indicative of the number of docking stations 104 in service by the healthcare facility or system, may include an action datum 710E indicative of the number of docking stations 104 currently needing action (e.g., maintenance), an upcoming action datum 712E indicative of a number of docking stations 104 expected to need action soon, an action requested datum 714E indicative of the number of docking stations 104 for which action has been requested, and an unavailability datum 716E indicative of the number of docking stations 104 that are currently unavailable for use (e.g., not functional). The docking station availability summary portion 834 may also include an available docking station datum 718E indicative of the number of docking stations 104 that are available for use and are neither in need of an action nor expected to be in need of action soon.

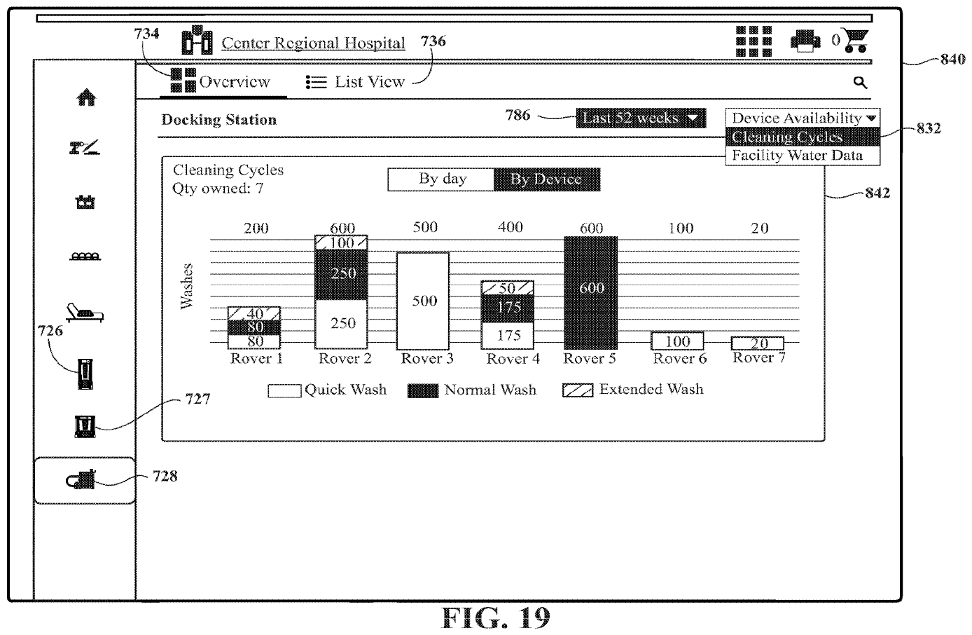

FIG. 19 illustrates another screen 840 that may be generated by the GUI responsive to selection of “Cleaning Cycle” in the navigational element 832. The screen 840 may generally be configured to indicate aggregate cleaning cycle data across the fleet of rovers 102. To this end, the screen 840 may include a bar chart 842 indicative the number of each of one or more types of cleaning cycles that have performed on each of the rovers 102 over a given period, which may be set with a user-interactive period selector 786 present on the screen 840.

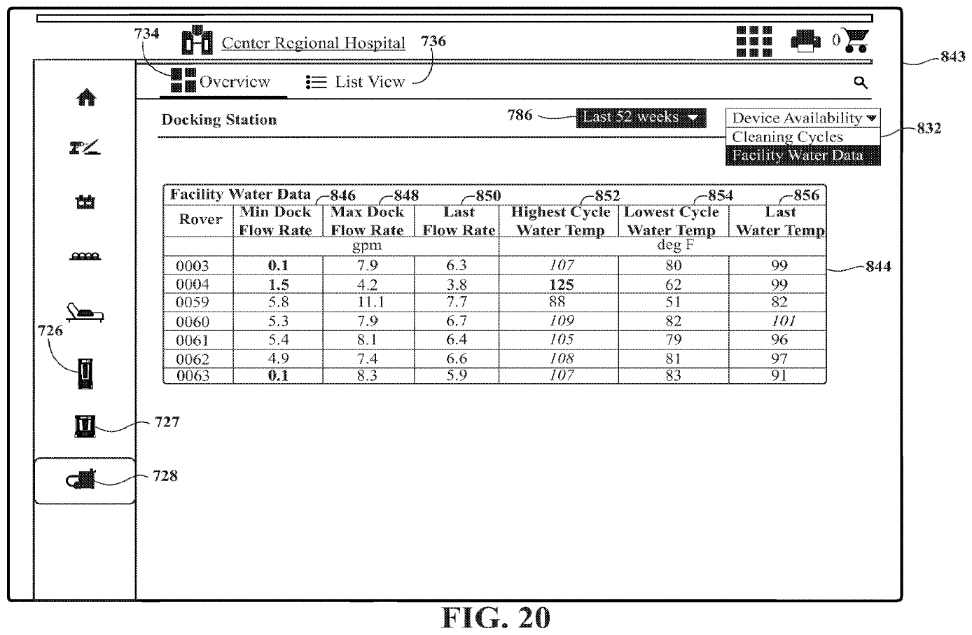

FIG. 20 illustrates a further screen 843 that may be generated by the GUI responsive to selection of “Facility Water Data” in the navigational element 832. The screen 843 may include a table 844 indicating several cleaning cycle datums for each of the rovers 102 relative to a given facility. For example and without limitation, the cleaning cycle datums may include one or more of a minimum flow rate datum 846 indicative of a minimum flow rate experienced by each rover 102 during a cleaning cycle over the set period; a maximum flow rate datum 848 indicative of a maximum flow rate experienced by each rover 102 during a cleaning cycle over the set period; a last flow rate datum 850 indicative of a last flow rate experienced by each rover 102 during a cleaning cycle over the set period; a highest water temperature datum 852 indicative of a highest water temperature experienced by each rover 102 during a cleaning cycle over the set period; a lowest water temperature datum 854 indicative of a lowest water temperature experienced by each rover 102 during a cleaning cycle over the set period; and a last water temperature datum 856 indicative of a last water temperature experienced by each rover 102 during a cleaning cycle over the set period.

The present disclosure provides systems and methods for managing a fleet of rovers 102 owned by a health care facility or system in a manner that is unconventional in the industry. In particular, unlike the previous method of noting a cleaning history of each system on whiteboards or other physical tracking sheet, which results in disjointed and out of date data that fails to consider a fleet of medical devices as a whole, the present disclosure describes systems and methods that incorporate a specific combination of unconventional features, such as leveraging docking stations used to clean the rovers as edge devices for allowing communication between the rovers and a remote central processing system, for implementing centralized management and notification processes for the fleet of rovers 102. Such processes may be configured to monitor the activity of a fleet of rovers as a whole to dynamically update maintenance and usage schedules for the fleet in real time, promoting both the longevity of the individual rovers and efficient use of the fleet, and to provide a complete and up to date view of the fleet that was not obtainable with previous methods. The systems and methods may also be configured to generate a notification to the person best suited to take action based on such monitoring so as to enable fast service and informed decision making.

In general, the routines executed to implement aspects of the foregoing description, whether implemented as part of an operating system or a specific application, component, program, object, module or sequence of instructions, or even a subset thereof, may be referred to herein as “computer program code,” or simply “program code.” Program code may comprise computer readable instructions that are resident at various times in various memory and storage devices in a computer and that, when read and executed by one or more processors in a computer, cause that computer to perform the operations necessary to execute operations and/or elements embodying the various aspects of the description. Computer readable program instructions for carrying out operations of the various aspects of the description may be, for example, assembly language or either source code or object code written in any combination of one or more programming languages.

The program code embodied in any of the applications/modules described herein may be capable of being individually or collectively distributed as a program product in a variety of different forms. In particular, the program code may be distributed using a computer readable storage medium having computer readable program instructions thereon for causing a processor to carry out aspects of the description.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include random access memory (RAM), read-only memory (ROM), erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions/acts specified in the flowcharts, sequence diagrams, and/or block diagrams. The computer program instructions may be provided to one or more processors such that the instructions, which execute via the one or more processors, cause a series of computations to be performed to implement the functions and/or acts specified in the flowcharts, sequence diagrams, and/or block diagrams described herein.

In certain alternatives, the functions and/or acts specified in the flowcharts, sequence diagrams, and/or block diagrams may be re-ordered, processed serially, and/or processed concurrently without departing from the scope of the invention. Moreover, any of the flowcharts, sequence diagrams, and/or block diagrams may include more or fewer blocks than those illustrated herein.

The terminology used herein is for the purpose of describing particular examples only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes,” “having,” “has,” “with,” “comprised of,” or variants thereof are used in either the detailed description or the claims, such terms are intended to be inclusive in a manner similar to the term “comprising.”

While a description of various examples has been provided and while these examples have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

Clause 1. A method of managing a medical waste collection unit including a canister for holding medical waste, wherein the medical waste collection unit is couplable with a docking station for emptying and cleaning the canister, the method comprising: resetting a counter or a timer for the medical waste collection unit in response to the canister of the medical waste collection unit being cleaned by the docking station; indexing the counter or initiating the timer in response to each instance the medical waste collection unit is operated to draw medical waste into the canister using a vacuum source; comparing the counter to a preset value or the timer to a first preset duration since a previous cleaning of the canister by the docking station; and based on the comparison, triggering a notification that directs a user to cause the docking station to clean the medical waste collection unit.

Clause 2. The method of clause 1, further comprising resetting the counter or the timer in response to the canister of the medical waste collection unit being cleaned in an extended cleaning cycle in which fluid from the docking station is sprayed within the canister for a duration greater than a second preset duration associated with another cleaning cycle of the docking station, wherein the preset value defines a number of extended cleaning cycles or the first preset duration defines a duration since a previous extended cleaning cycle of the canister, and wherein the notification directs the user to cause the docking station to clean the medical waste collection unit in the extended cleaning cycle.

Clause 3. The method of clause 2, further comprising not resetting the counter or the timer in response to the canister of the medical waste collection unit being cleaned by the docking station in the another cleaning cycle.

Clause 4. The method of clause 3, further comprising: determining a ratio of a number of times the canister is cleaned by the docking station in the another cleaning cycle to a number of times the canister is cleaned by the docking station in the extended cleaning cycle; comparing the ratio to a preset ratio; and based on the comparison, triggering the notification.

Clause 5. The method of any one of clauses 2-4, further comprising displaying on one or more displays a graphical user interface including one or more user selectable options associated with the extended cleaning cycle, the one or more user selectable options being configured to set at least one of a fill level, canister soak time, detergent volume, detergent addition timing, water pressure, water temperature, ingredient addition, or ultraviolet light activation to be implemented in a subsequent instance that the medical waste collection unit is cleaned in the extended cleaning cycle.

Clause 6. The method of any one of clauses 1-5, further comprising displaying on one or more displays a graphical user interface including one or more user selectable options for setting the preset value and/or the first preset duration.

Clause 7. The method of clause 5 or 6, wherein the one or more displays comprise a display of the medical waste collection unit and/or a display of a personal computing device remote from the medical waste collection unit.

Clause 8. The method of any one of clauses 1-7, further comprising triggering display of the notification on a display of the medical waste collection unit.

Clause 9. The method of clause 8, further comprising triggering display of the notification on the display of the medical waste collection unit such that the notification is displayed while the medical waste collection unit is not being operated to collect medical waste, wherein the notification directs the user to cause the docking station to clean the medical waste collection unit prior to commencing another waste collection operation.

Clause 10. The method of clause 8 or 9, further comprising triggering display of the notification on the display of the medical waste collection unit such that the notification is displayed responsive to the medical waste collection unit exiting an idle state.

Clause 11. The method of any one of clauses 7-10, further comprising triggering display of the notification on the display of the medical waste collection unit such that the notification is displayed responsive to the medical waste collection unit being docked with the docking station.

Clause 12. The method of clause 11, wherein triggering display of the notification on the display of the medical waste collection unit such that the notification is displayed responsive to the medical waste collection unit being docked with the docking station comprises communicating, by a remote processing system, notification data to the docking station over one or more computer networks responsive to the medical waste collection unit being docked with the docking station, the notification data causing the docking station to instruct the medical waste collection unit to display the notification.

Clause 13. The method of clause 12, further comprising: receiving, by the remote processing system, a status update from the docking station indicative of a docked status of the medical waste collection unit responsive to the medical waste collection unit being docked with the docking station; and communicating, by the remote processing system, the notification data to the docking station responsive to receiving the status update.

Clause 14. The method of clause 13, further comprising: receiving, by the remote processing system, a request for a state of the medical waste collection unit from a personal computing device remote from the medical waste collection unit; and responsive to receiving the request, communicating, by the remote processing system, status data to the personal computing device indicating that the medical waste collection unit is docked to the docking station.

Clause 15. The method of any one of clauses 1-14, further comprising, responsive to the canister of the medical waste collection unit being cleaned by the docking station, receiving, by a remote processing system, updated cleaning data for the medical waste collection unit from the docking station over one or more computer networks.

Clause 16. The method of clause 15, wherein the updated cleaning data comprises one or more of the counter for the medical waste collection unit, the timer for the medical waste collection unit, or at least one characteristic of a last cleaning cycle performed on the medical waste collection unit.

Clause 17. The method of clause 15 or 16, wherein the updated cleaning data is communicated from the medical waste collection unit to the docking station responsive to the medical waste collection unit being docked to the docking station and/or to the canister of the medical waste collection unit being cleaned by the docking station.

Clause 18. The method of any one of clauses 1-17, wherein the medical waste collection unit is a first medical waste collection unit, and further comprising: generating a ranked list of relative cleanliness of a fleet of medical waste collection units including the first medical waste collection unit based on a counter indexed or a timer initiated for each of the medical waste collection units in response to each instance the medical waste collection unit is operated to draw medical waste into a canister of the medical waste collection unit using a vacuum source; and providing the ranked list for display on the medical waste collection units and/or a personal computing device remote from the medical waste collection units.

Clause 19. The method of clause 18, wherein a display is mounted on a mobile chassis of the first medical waste collection unit, and, optionally, wherein data indicative of the ranked list of relative cleanliness is transmitted from a remote processing system to the medical waste collection unit over one or more computer networks to be displayed on the chassis-mounted display.

Clause 20. The method of clause 19, wherein the data is pushed to each of the medical waste collection units of the fleet to be selectively viewable on a chassis-mounted display of the medical waste collection unit.

Clause 21. The method of any one of clauses 1-20, further comprising triggering display of the notification on a display of a personal computing device remote from the medical waste collection unit.

Clause 22. A method of managing a fleet of medical waste collection units each including a canister for holding medical waste, wherein the medical waste collection units are interchangeably couplable with a docking station for emptying and cleaning the canisters, the method comprising: receiving first usage data from a first of the medical waste collection units indicative of a duration in which the first medical waste collection unit is operated to draw medical waste into the canister of the first medical waste collection unit by a vacuum source; receiving second usage data from a second of the medical waste collection units indicative of a duration in which the second medical waste collection unit is operated to draw medical waste into the canister of the second medical waste collection unit by a vacuum source; comparing the first usage data with the second usage data to determine relative usage data for the first medical waste collection unit and the second medical waste collection unit; comparing the relative usage data to a preset relative usage threshold; and based on the comparison, triggering a notification.

Clause 23. The method of clause 22, wherein the notification directs a user to use a less frequently used one of the first medical waste collection unit and the second medical waste collection unit in a subsequent medical waste collection procedure.

Clause 24. The method of clause 22 or 23, further comprising triggering display of the notification on a first display mounted on a chassis of the first medical waste collection unit in response to the first medical waste collection unit exiting an idle state and/or on a second display mounted on a chassis of the second medical waste collection unit in response to the second medical waste collection unit exiting the idle state.

Clause 25. The method of clause 24, further comprising: receiving, by a processing system remote from the first and/or second medical waste collection units, a status update from one of the first and second medical waste collection units indicative of a non-idle status of the one of the first and second medical waste collection units responsive to the one of the first and second medical waste collection units exiting the idle state; and communicating, by the remote processing system, notification data to the one of the first and second medical waste collection units responsive to receiving the status update, the notification data causing the one of the first and second medical waste collection units to display the notification.

Clause 26. The method of clause 24 or 25, further comprising transmitting, by a processing system remote from the first and/or second medical waste collection units, the relative usage data to the first and/or second medical waste collection units over one or more computer networks to be selectively viewable on the first and/or second displays, respectively.

Clause 27. The method of any one of clauses 24-26, further comprising transmitting, by a processing system remote from the first and/or second medical waste collection units, the relative usage data to the first medical waste collection unit over one or more computer networks in response to the first medical waste collection unit being docked with the docking station and/or to the second medical waste collection unit over one or more computer networks in response to the second medical waste collection unit being docked with the docking station.

Clause 28. The method of any one of clauses 22-27, further comprising triggering display of the notification on a display of a more frequently used one of the first and second medical waste collection units.

Clause 29. The method of any one of clauses 22-28, further comprising triggering display of the notification on a display of a personal computing device remote from the first and second medical waste collection units.

Clause 30. The method of any one of clauses 22-29, further comprising: generating a ranked list of relative usage of the first medical waste collection unit, the second medical waste collection unit, and additional medical waste collection units of the fleet; and enabling selective display of the ranked list of relative usage.

Clause 31. The method of any one of clauses 22-30, further comprising: determining a relative usage ratio based on the first usage data and the second usage data; comparing the relative usage ratio to a preset usage ratio; and based on the comparison, triggering display of the notification.

Clause 32. The method of clause 31, wherein the preset usage ratio is at least one hundred fifty percent.

Clause 33. The method of any one of clauses 22-32, wherein the notification is further configured to direct the user to dock a more frequently used one of the first and second medical waste collection units with the docking station for cleaning.

Clause 34. A method of managing a medical waste collection unit including a canister for holding medical waste and an aerosol evacuation unit including a filter with an expected filter life, the method comprising: resetting a filter timer for the medical waste collection unit in response to a filter change event; initiating the filter timer in response to each instance the medical waste collection unit is operated to draw fluid through the filter; and based on the filter timer and the expected filter life, triggering display of a notification on a display of a personal computing device remote from the medical waste collection unit that directs a user to replace the filter.

Clause 35. The method of clause 34, further comprising: determining a filter usage threshold based on a preset percentage of the expected filter life; and triggering display of the notification on the personal computing device based on the filter timer and the filter usage threshold.

Clause 36. The method of clause 35, further comprising displaying a graphical user interface on the display of the personal computing device and/or on a display of the medical waste collection unit, the graphical user interface including one or more user selectable options for setting the expected filter life and/or the preset percentage.

Clause 37. The method of any one of clauses 34-36, wherein the aerosol evacuation unit comprises an aerosol sensor configured to generate a signal indicative whether an aerosol is being drawn through the filter, and further comprising initiating the filter timer in response to each instance the signal from aerosol sensor indicates that an aerosol is being drawn through the filter.

Clause 38. The method of clause 37, wherein the aerosol sensor comprises a smoke sensor and the filter comprises a smoke filter, optionally an ULPA filter.

Clause 39. The method of any one of clauses 34-36, wherein the filter comprises a smoke filter, optionally an ULPA filter.

Clause 40. The method of any one of clauses 34-39, wherein the filter comprises a HEPA filter.

Clause 41. The method of any one of clauses 34-40, further comprising providing at least one of the filter timer or a remaining filter life for presentation on the display on the personal computing device and/or a display of the medical waste collection unit.

Clause 42. The method of any one of clauses 34-41, wherein the medical waste collection unit is couplable with a docking station for emptying and cleaning the canister, and further comprising: receiving, at a processing system remote from the medical waste collection unit, filter usage data for the medical waste collection unit from the docking station over one or more computer networks responsive to the medical waste collection unit docking with the docking station; aggregating, by the remote processing system, the received filter usage data with filter usage data from other medical waste collection units to generate aggregate filter use data; and communicating, by the remote processing system, aggregate filter usage data to the personal computing device for presentation on the personal computing device display.

Clause 43. The method of clause 42, wherein the filter usage data comprises the filter timer and/or the expected filter life.

Clause 44. The method of clause 42 or 43, wherein the filter usage data comprises a profile assigned to each usage of the filter during a medical procedure, the profile including one or more datums selected from the group consisting of date of use, time of use, medical specialty, type of procedure, medical waste collection unit part number, and filter part number.

Clause 45. A method of managing a fleet of medical waste collection units each including a canister for holding medical waste and a manifold receiver configured to receive replaceable manifolds, the method comprising: receiving usage data from each of the medical waste collection units; aggregating the received usage data to generate aggregate use data indicative of usage characteristics across the fleet of medical waste collection units; and providing the aggregate usage data for display on a display of a personal computing device remote from the medical waste collection units.

Clause 46. The method of clause 45, wherein each of the medical waste collection units comprises a fluid measuring system, and the usage data from each medical waste collection unit comprises a fluid disposal volume measured by the fluid measuring system of the medical waste collection unit for each usage of the replacement manifolds during a medical procedure.

Clause 47. The method of any one of clause 46, further comprising: determining a fluid disposal cost based on the fluid disposal volumes and a weight-based disposal cost conversion; comparing the fluid disposal cost and an operational cost of the medical waste collection units to determine a cost savings; and providing the cost savings for display on the remote personal computing device.

Clause 48. The method of any one of clauses 45-47, wherein the usage data from each medical waste collection unit comprises a profile assigned by the medical waste collection unit to each usage of the replacement manifolds during a medical procedure.

Clause 49. The method of clause 48, wherein the profile assigned to each usage of the replaceable manifolds includes one or more datums selected from the group consisting of date of use, time of use, medical specialty, type of procedure, medical waste collection unit type and/or model number, medical waste collection unit unique identifier, and manifold type and/or model number, and manifold unique identifier.

Clause 50. The method of any one of clauses 45-49, wherein the medical waste collection units are interchangeably couplable with a docking station for emptying and cleaning the canister of each medical waste collection unit, and further comprising receiving, at a processing system remote from the medical waste collection units and over one or more computer networks, the usage data for each of the medical waste collection units from the docking station responsive to the medical waste collection unit docking with the docking station.

Clause 51. A method of managing a fleet of medical waste collection units each including a canister for holding medical waste, the method comprising: receiving error code data for each of the medical waste collection units, the error code data indicating one or more error codes generated by the medical waste collection unit based on abnormalities identified during operation of the medical waste collection unit; aggregating the received error code data to generate aggregate error code data that is indicative of diagnostic patterns across the fleet of medical waste collection units; and provide the aggregate error code data for display on a personal computing device remote from the medical waste collection units.

Clause 52. The method of clause 51, wherein the error code data from each of the medical waste collection units comprises one or more datums selected from the group consisting of date of use, time of use, medical specialty, type of procedure, medical waste collection unit type and/or model, and medical waste collection unit unique identifier.

Clause 53. The method of clause 51 or 52, wherein the medical waste collection units are interchangeably couplable with a docking station for emptying and cleaning the canister of each of the medical waste collection units, and further comprising receiving, at a processing system remote from the medical waste collection units, the error code data for each of the medical waste collection units from the docking station responsive to the medical waste collection unit docking with the docking station.

Clause 54. A method of managing operation of a medical waste collection unit including a canister for holding medical waste and being configured to be removably coupled to a docking station for emptying and cleaning the canister of the medical waste collection unit, the method comprising: responsive to the medical waste collection unit being docked to the docking station for running a cleaning cycle on the canister of the medical waste collection unit, receiving, at a processing system remote from the medical waste collection unit and over one or more computer networks, a status update from the docking station indicating that the medical waste collection unit is docked to the docking station; responsive to receiving the status update, determining, by the remote processing system, that a notification should be triggered; and responsive to determining that the notification should be triggered, triggering, by the remote processing system, the notification.

Clause 55. The method of clause 44, furthering comprising triggering the notification to be displayed on a display of the medical waste collection unit by sending notification data to the docking station over the one or more computer networks that causes the docking station to instruct the medical waste collection unit to display the notification, and/or triggering the notification on a display of a personal computing device remote from the medical waste collection unit.

Clause 56. The method of clause 54 or 55, further comprising: responsive to the medical waste collection unit being docked to the docking station for cleaning the canister of the medical waste collection unit, receiving, at the remote processing system, operational data relating to activity of the medical waste collection unit; and determining, by the remote processing system, that the notification should be triggered based on the operational data.

Clause 57. The method of clause 56, further comprising: responsive to receiving the operational data, aggregating the operational data with operational data received from other medical collection units when docked to a docking station to generate aggregate operational data; and determining that the notification should be triggered based on the aggregate operational data.

Clause 58. The method of clause 57, further comprising providing the operational data for the medical waste collection unit and/or the aggregated operational data for presentation on a display of a personal computing device remote from the medical waste collection unit.

Clause 59. The method of any one of clauses 54-58, wherein the operational data for the medical waste collection unit comprises one or more of cleaning related data for the medical waste collection unit, error code data for the medical waste collection unit, filter usage data for the medical waste collection unit, vacuum pump runtime data for the medical waste collection unit, manifold usage data for the medical waste collection unit, or fluid disposal volume data for the medical waste collection unit.

Clause 60. The method of any one of clauses 54-59, further comprising: responsive to the cleaning cycle being run on the medical waste collection unit, receiving, at the remote processing system, cleaning data for the medical waste collection unit from the docking station; determining, by the remote processing system, that a cleaning related notification should be displayed on the medical waste collection unit based on the cleaning data; and triggering, by the remote processing system, display of the cleaning related notification on a display the medical waste collection unit responsive to a subsequent docking of the medical waste collection unit with the docking station.

Clause 61. A method of managing operation of a fleet of medical waste collection units including a canister for holding medical waste and being configured to be removably coupled to a docking station for emptying and cleaning the canister of the medical waste collection unit, the method comprising: responsive to the medical waste collection unit being docked to the docking station for running a cleaning cycle on the canister of the medical waste collection unit, receiving, at a processing system remote from the medical waste collection unit and over one or more computer networks, operational data relating to activity of the medical waste collection unit from the docking station; responsive to receiving the operational data, aggregating, by the remote processing system, the operational data with operational data received from the other medical collection units when docked to a docking station to generate aggregate operational data; and providing, by the remote processing system, the operational data for the medical waste collection unit and/or the aggregated operational data for presentation on a display of a personal computing device remote from the medical waste collection units.

Clause 62. The method of clause 61, wherein the operational data for the medical waste collection unit comprises one or more of cleaning related data for the medical waste collection unit, error code data for the medical waste collection unit, filter usage data for the medical waste collection unit, vacuum pump runtime data for the medical waste collection unit, manifold usage data for the medical waste collection unit, or fluid disposal volume data for the medical waste collection unit.

Clause 63. A computer program product stored on non-transitory memory and comprising computer-executable instructions configured, upon execution by at least one controller or processor, to implement the method of any one of clauses 1-62.

Clause 64. At least one controller or processor configured to implement the method of any one of clauses 1-62.

Clause 65. A system for managing operation of a medical waste collection unit including a canister for holding medical waste, the system comprising: a remote processing system; and a docking station for cleaning the canister of the medical waste collection unit, the docking station being in communication with the remote processing system over one or more computer networks and configured to be removably coupled with the medical waste collection unit to form a fluid connection and a data connection with the medical waste collection unit, wherein responsive to being coupled to the medical waste collection unit, the docking station is configured to: perform a cleaning cycle in which fluid for being sprayed into the canister of the medical waste collection unit is supplied from the docking station to the medical waste collection unit over the fluid connection, communicate a status update to the remote processing system indicative of a docked status of the medical waste collection unit; receive notification data from the remote processing unit responsive to the status update indicative of whether a notification should be displayed on the medical waste collection unit; and based on the notification data, trigger the medical waste collection unit to display the notification using the data connection.

Clause 66. The system of clause 65, wherein responsive to being coupled to the medical waste collection unit, the docking station is further configured to: receive operational data relating to activity of the medical waste collection unit over the data connection; and communicate the operational data to the remote processing system over the one or more computer networks, wherein the remote processing system is configured to generate the notification data based on the operational data for the medical waste collection unit.

Clause 67. The system of clause 66, wherein the remote processing system is configured to: responsive to receiving the operational data, aggregate the operational data with operational data received from another medical waste collection unit when docked to the docking station to generate aggregate operational data; and trigger the notification data based on the aggregate operational data.

Clause 68. The system of clause 67, wherein the remote processing system is configured to provide the operational data for the medical waste collection unit and/or the aggregated operational data for presentation on a display of a personal computing device remote from the medical waste collection units.

Clause 69. The system of any one of clauses 65-68, wherein the operational data for the medical waste collection unit comprises one or more of cleaning related data for the medical waste collection unit, error code data for the medical waste collection unit, filter usage data for the medical waste collection unit, vacuum pump runtime data for the medical waste collection unit, manifold usage data for the medical waste collection unit, or fluid disposal volume data for the medical waste collection unit.

Clause 70. The system of any one of clauses 65-69, wherein responsive to the cleaning cycle being run on the medical waste collection unit, the docking station is configured to communicate cleaning related data for the medical waste collection unit to the remote processing system over the one or more computer networks, and wherein the remote processing system is configured to trigger display of a cleaning related notification on the medical waste collection unit on a subsequent docking of the medical waste collection unit with the docking station based on the cleaning related data.

Clause 71. A system for managing a fleet of medical waste collection units each including a canister for collecting medical waste, the system comprising: a remote processing system; and a docking station for cleaning the canisters of the medical waste collection units, the docking station being in communication with the remote processing system over one or more computer networks and configured to be removably coupled with each of the medical waste collection units to form a fluid connection and a data connection with the medical waste collection unit, wherein responsive to being coupled to one of the medical waste collections units, the docking station is configured to: perform a cleaning cycle in which fluid for being sprayed into the canister of the medical waste collection unit is supplied from the docking station to the medical waste collection unit over the fluid connection, receive operational data from the medical waste collection unit over the data connection, and transmit the operational data to the remote processing system; and wherein the remote processing system is configured to: aggregate the operational data for each of the medical waste collection units that is received from the docking stations; and provide the aggregate operational data for display on the medical waste collection units and/or on a personal computing device remote from the medical waste collection units.

Clause 72. The system of clause 71, wherein the remote processing system is configured to transmit the aggregate operational data to the docking station for being presented on a display of each of the medical waste collection units responsive to the medical waste collection unit being subsequently coupled to the docking station.

Clause 73. The system of clause 71 or 72, wherein the operational data for the medical waste collection unit comprises one or more of cleaning related data for the medical waste collection unit, error code data for the medical waste collection unit, filter usage data for the medical waste collection unit, vacuum pump runtime data for the medical waste collection unit, manifold usage data for the medical waste collection unit, or fluid disposal volume data for the medical waste collection unit.

1. A method of maintaining a medical waste collection unit including a canister for collecting medical waste produced during a surgical procedure, the method comprising:
transporting the medical waste collection unit with the collected medical waste to a docking station, the docking station including a supply line coupled to a first interface for establishing a fluid supply connection with the medical waste collection unit when docked with the docking station, a discharge line coupled to a second interface for establishing a fluid discharge connection with the medical waste collection unit when docked with the docking station, a first communications interface for establishing a first data connection with the medical waste collection unit when docked to the docking station, and a second communications interface for establishing a second data connection with a remote processing system;
docking the medical waste collection unit with the docking station to form the fluid supply connection, the fluid discharge connection, and the first data connection;
performing a cleaning cycle on the medical waste collection unit in which cleaning fluid is supplied from the docking station supply line to the medical waste collection unit through the fluid supply connection and sprayed into the canister, and in which waste material is discharged from the canister to the docking station discharge line through the fluid discharge connection;
communicating, from the docking station and through the second data connection, a status update to the remote processing system indicative of a docked status of the medical waste collection unit;
receiving, at the docking station and through the second data connection, notification data from the remote processing system that is responsive to the status update, the notification data indicating a maintenance notification to be displayed on the medical waste collection unit; and
triggering, by the docking station and through the first data connection, the medical waste collection unit to display a maintenance notification based on the notification data.

2. The method of claim 1, comprising:
tracking, by the remote processing system, a metric for determining whether to trigger the maintenance notification on the medical waste collection unit;
receiving, by the remote processing system, operational data of the medical waste collection unit, the operational data indicating surgical procedure data of the medical waste collection unit and/or cleaning cycle data of the medical waste collection unit;
determining, by the remote processing system, a notification threshold based on the operational data; and
generating the notification data based on the metric and the notification threshold.

3. The method of claim 2, wherein the surgical procedure data comprises at least one of error code data, filter usage data, vacuum pump runtime data, manifold usage data, medical waste volume data, procedure type data, and patient data.

4. The method of claim 2, wherein the cleaning cycle data comprises at least one of water temperature data, water pressure data, detergent data, and cleaning cycle type data.

5. The method of claim 2, comprising:
capturing, by an imaging device supported by a device cradle coupled to the canister, a video feed of the canister and the waste material disposed in the canister as a vacuum source of the medical waste collection unit is drawing waste material into the canister;
analyzing, by the imaging device or the remote processing system, image frames of the video feed to determine vision data indicating at least one of a degree of occlusion, a composition of the waste material, and a storage time of the waste material; and
determining, by the remote processing system, the notification threshold based on the vision data.

6. The method of claim 2, comprising:
aggregating, by the remote processing system, the operational data of the medical waste collection unit with operational data received from another medical waste collection unit when docked to the docking station to generate aggregate operational data; and
generating, by the remote processing system, the notification data based on the aggregate operational data.

7. The method of claim 6, comprising providing the operational data for the medical waste collection unit and/or the aggregate operational data for presentation on a display of a personal computing device remote from the medical waste collection units.

8. The method of claim 6, comprising:
generating a ranked list of relative cleanliness of a fleet of medical waste collection units including the medical waste collection unit and the another medical waste collection unit based on the aggregate data; and
providing the ranked list for display on the medical waste collection unit and/or a personal computing device remote from the medical waste collection units.

9. The method of claim 6, comprising:
determining, by the remote processing system, that the medical waste collection unit is more frequently used than the another medical waste collection unit based on the aggregate data; and
responsive to determining that the medical waste collection unit is more frequently used, communicating, by the remote processing system and over the second data connection, further notification data indicating a usage notification to be displayed on the medical waste collection unit that directs a user to use the another medical waste collection unit in a subsequent procedure; and
triggering, by the docking station and through the first data connection, the medical waste collection unit to display the usage notification based on the further notification data.

10. The method of claim 1, wherein the maintenance notification indicates to perform an extended cleaning cycle on the medical waste collection unit in which the cleaning fluid from the docking station supply line is sprayed within the canister for a duration greater than a duration associated with another cleaning cycle available to the medical waste collection unit from the docking station.

11. The method of claim 10, comprising:
tracking, by the remote processing system, a cleaning cycle metric for determining whether to trigger the maintenance notification indicating to perform the extended cleaning cycle;
performing the extended cleaning cycle on the medical waste collection unit;
responsive to performing the extended cleaning cycle on the medical waste collection unit, resetting, by the remote processing system, the cleaning cycle metric; and
performing the another cleaning cycle on the medical waste collection unit, wherein the cleaning cycle metric is not reset responsive to performing the another cleaning cycle.

12. The method of claim 10, wherein the cleaning cycle metric comprises a ratio of a number of times the canister is cleaned by the docking station in the another cleaning cycle to a number of times the canister is cleaned by the docking station in the extended cleaning cycle.

13. The method of claim 1, wherein the performance of the cleaning cycle and the communication of the status update occur during a first docking of the medical waste collection unit with the docking station, and the triggering, by the docking station, of the medical waste collection unit to display the maintenance notification occurs during a subsequent docking of the medical waste collection unit with the docking station.

14. The method of claim 1, comprising:
responsive to performing the cleaning cycle, resetting, by the medical waste collection unit, a locally stored cleaning cycle metric for determining whether to display a notification indicating to perform further cleaning cycle;
comparing, by the medical waste collection unit, the locally stored cleaning cycle metric to a cleaning notification threshold; and
displaying, by the medical waste collection unit, the notification indicating to perform the further cleaning cycle based on the comparison.

15. The method of claim 14, wherein the cleaning notification threshold is dynamically adjusted by the medical waste collection unit based on operational data of the medical waste collection unit recorded during at least one surgical procedure.

16. The method of claim 1, wherein the medical waste collection unit includes an aerosol evacuation unit including a filter, and the maintenance notification indicates to replace the filter.

17. The method of claim 16, wherein the aerosol evacuation unit comprises an aerosol sensor configured to generate a signal indicative whether an aerosol is being drawn through the filter, and comprising:
tracking, by the remote processing system, a filter metric for determining whether to indicate to replace the filter based on the signal; and
generating, by the remote processing system, the notification data based on the filter metric.

18. The method of claim 17, wherein the aerosol sensor comprises a smoke sensor and the filter comprises a smoke filter.

19. The method of claim 1, comprising triggering, by the remote processing system, display of a second notification corresponding to the notification on a personal computing device remote from the medical waste collection unit.

20. The method of claim 1, comprising causing, by the medical waste collection unit, display of the notification during the cleaning cycle.

21.-48. (canceled)
